(19) United States
(12) Patent Application Publication
(10) Pub. No.: US 2026/0283718 A1
(43) Pub. Date: Sep. 24, 2026

(54) MR-COMPATIBLE ROBOTIC CANTILEVER PLATFORM

(71) Applicants: GEORGIA TECH RESEARCH CORPORATION, Atlanta, GA (US); THE JOHNS HOPKINS UNIVERSITY, Baltimore, MD (US)

(72) Inventors: Yue CHEN, Atlanta, GA (US); Yifan WANG, Atlanta, GA (US); Anthony GUNDERMAN, Atlanta, GA (US); Henry HALPERIN, Baltimore, MD (US); Mirko HROVAT, Baltimore, MD (US); Aravindan KOLANDAIVELU, Baltimore, MD (US); Ehud SCHMIDT, Baltimore, MD (US)

(21) Appl. No.: 19/476,581

(22) PCT Filed: Apr. 17, 2024
(86) PCT No.: PCT/US2024/025003

§ 371 Date: Oct. 17, 2025

Related U.S. Application Data
(60) Provisional application No. 63/496,580, filed on Apr. 17, 2023.

(51) Int. Cl. A61B 34/30; A61B 34/20

(52) U.S. Cl. CPC A61B 34/30; A61B 34/20; A61B 2034/2051

(57) ABSTRACT

Exemplary system and method are disclosed that employ an MR-compatible robotic system having a cantilever arm member comprising an elongated beam to extend into the available clearance space of the bore of an MRI scanner when the patient is inside to position interventional and scanning assisted instruments during an MRI scan. The exemplary MR-compatible robotic system can provide multiple direction of motion in the scanner bore with accuracy of less than 2 mm position error for a heavy payload (e.g., greater than or up to 5 kg) while being made of MR-compatible non-metal material, e.g., PLA, Nylon, fiberglass, Acrylic and other plastics. The MR-compatible robotic system is portable in being movable and not-permanently fixed to the MR scanner to be compatible with any closed bore MR scanners (as well as any open-bore MR scanners).

### RELATED APPLICATION

This PCT application claims priority to, and the benefit of, U.S. Provisional Patent Application No. 63/496,580, filed Apr. 17, 2023, which is incorporated by reference herein in its entirety.

### GOVERNMENT LICENSE RIGHTS

This invention was made with government support under grant no. HL166053 awarded by the National Institutes of Health. The government has certain rights in the invention.

### BACKGROUND

There is a benefit to making the bore of an MRI scanner more physically accessible despite their limited physical size and presence of high magnetic field. A magnetic resonance imaging (MRI) machine performs imaging using a combination of strong static (DC) magnetic fields, magnetic field gradients (fields in the tens of KHz range), and radio waves (fields in the tens to hundreds of MHz). At certain magnetic field strengths, issues can arise with ferromagnetic, paramagnetic, or electrically conducting materials positioned inside or near the MRI machine due to the coupling between these objects and the fields within the MRI scanner.

Among these ferromagnetic objects are implantable cardioverter-defibrillators (ICDs), which are battery-powered devices that are usually implanted in the left chest wall over the rib cage. Approximately 150,000 ICD implantations are carried out in the US annually and about half of the people with implanted ICDs require an MRI scan for diagnostic purposes. Cardiac MRI (CMR) is of increasing interest in the ICD patient population due to its ability to produce high-resolution images for detecting myocardial scarring which can lead to VT and VF. However, obtaining high-quality images that characterize the scar's 3D topography can be challenging due to the ferromagnetic transformer present in the ICD.

One protocol for manipulating MRI image quality with patients having ICDs is to use a shim coil, which can improve magnetic field homogeneity and correct ICD-related distortion within the heart. However, this strategy typically requires several iterations of repositioning the coil within the small opening of the MRI scanner to a region of interest in order to collect acceptable images, resulting in a heavily inefficient and labor-intensive process. Manual adjustment, while time consuming, also cannot achieve scanning while accounting for motion between each adjustments.

Moreover, certain interventional MRI procedures, such as MR-assisted ablation, can entail positioning instrumentation or instrumentation-assisting components inside the “scanner bore” of the MRI scanner during high magnetic field operation. However, these procedures can face similar challenges, such as requiring movement within the narrow opening of the machine.

Open MRI scanner systems employ a different physical configuration of the bore to allow more physical access. This does not address the issue for existing MRI scanners.

Thus, there is a benefit to improving scanning via MRI machines.

### SUMMARY

Exemplary system and method are disclosed that employ an MR-compatible robotic system having a cantilever arm member comprising an elongated beam to extend into the available clearance space of the bore of an MRI scanner when the patient is inside to position interventional and scanning assisted instruments during an MRI scan. The exemplary MR-compatible robotic system can provide multiple direction of motion in the scanner bore with accuracy of less than 2 mm position error for a heavy payload (e.g., greater than or up to 5 kg) while being made of MR-compatible non-metal material, e.g., PLA, Nylon, fiberglass, Acrylic and other plastics. The MR-compatible robotic system is portable in being movable and not-permanently fixed to the MR scanner to be compatible with any closed bore MR scanners (as well as any open-bore MR scanners).

The exemplary MR-compatible robotic system is actuated by pneumatically-driven motors equipped with optical quadrature encoders to provide closed-loop position control to provide scanning with real-time motion control while the patient is located within the bore. The closed-loop position control includes collision control that can directly and safely direct the tip of the cantilever arm member to the target region while halting for any sensed haptic feedback of contact of any portion of the cantilever arm or instrument with the bore, the patient, or any physical obstructions in the bore.

In an aspect, a motorized robotic system (e.g., an MR-compatible robotic system) is disclosed that include an arm member comprising an elongated beam (e.g., a cantilever beam) extending along a longitudinal axis and having a first end and a second end. The first end of the arm member can include an instrument (e.g., coil, ablation instrument, surgical device) configured to be at least partly positionable within an opening of an MRI scanner having a bore or elongated section, the bore or elongated section having a distal end and a proximal end, and the MRI scanner having the opening of the bore or elongated section at the proximal end. The motorized robotic system also includes a base assembly moveably coupled to the second end of the arm member, the base assembly having a motion unit comprising one or more motors (e.g., pneumatic, hydraulic, or piezoelectric motors) configured to, while a patient is located in the bore or elongated section, (i) move the arm member along the longitudinal axis from (a) a first position having the instrument outside of the bore or elongated section to (b) a second position within the bore or elongated section to place the instrument at or toward the distal end of the bore or elongated section and (ii) move the arm member in a horizontal axis perpendicular to the longitudinal axis and/or a vertical axis at the second position to position the instrument at or around the distal end of the bore or elongated section in relation to the patient.

In some aspects, the motorized robotic system further includes a controller, the controller being configured to detect an impact or contact of the instrument or arm member to a wall of the bore or elongated section, to the patient, or an object in the bore or elongated section. The base assembly, the arm member, and/or the instrument can include one or more sensors (e.g., a quadrature encoder) to provide spatial data to the controller associated with a current position of the arm member or the instrument (e.g., wherein the controller is configured to halt movement based on the detection).

In various aspects, the controller is further configured to: receive the spatial data from the one or more sensors; and control the one or more motors of the motion unit to move the arm member from the current position to a target position.

In some aspects, the controller is configured to: receive movement data associated with the patient; and control the one or more motors to move the arm member based on the patient movement data (e.g., to account for breathing).

In some aspects, the arm member and base assembly are substantially free of ferromagnetic materials. In some aspects, the arm member and base assembly are substantially free of paramagnetic materials. In some aspects, the arm member and base assembly are substantially free of ferromagnetic and paramagnetic materials. For example, the arm member and base assembly can comprise an MR-compatible material (e.g., selected from the group consisting of Polylactic acid (PLA), nylon, fiberglass, and acrylic).

In some aspects, the base assembly includes a counterweight system coupled to the arm member at or proximal to the second end. The counterweight system can be configured to be dynamically translatable along the longitudinal axis and/or the vertical axis to account for movement of the arm member (e.g., longitudinal translation).

In some aspects, the motion unit includes one or more lead screws operatively coupled to the one or more motors such that actuation of the one or more motors causes the lead screw to rotate such that the arm member translates relative to the base assembly.

In some aspects, the motion unit includes a first lead screw positioned along the longitudinal axis, a second lead screw positioned along the horizontal axis, and a third lead screw positioned along the vertical axis.

In some aspects, the base assembly is disposed on an adjustable carrier, wherein the adjustable carrier is configured to be maneuverable (e.g., using hydraulic or pneumatic actuation) about the horizontal and/or vertical axes to move the base assembly about a height and/or a center of the bore or elongated section.

In some aspects, the instrument includes a shim coil configured to generate a magnetic field to create a controlled magnetic field, the shim coil defining an electromagnet secured (e.g., fixedly or movably) to the first end of the arm member.

In some aspects, the instrument includes a second robotic assembly coupled to an ablation instrument (Radio-Frequency (RF) or thermal), an ultrasound probe (e.g., of the liver), a biopsy excising instrument, or a fluoroscopy instrument (or another interventional instrument).

In some aspects, the instrument includes an ablation instrument (RF or thermal), an ultrasound probe (e.g., of the liver), a biopsy excising instrument, or a fluoroscopy instrument (or another interventional instrument).

Also described herein are methods for positioning an instrument within an opening of an MRI scanner. In various aspects, the method includes: operating the motorized robot system discussed herein to move the instrument within the opening of the MRI scanner.

The present disclosure further provides methods for reducing artifacts in magnetic resonance imaging. In various aspects, the method includes: operating the motorized robot system discussed herein to position the shim coil proximate to a source of distortion (e.g., an implantable cardioverter defibrillator (ICD) or a pacemaker); and supplying a current to the shim coil to produce a controllably distorted magnetic field opposing a magnetic field of the source of distortion.

In some aspects, the method includes incrementally moving the shim coil and/or varying the current to the shim coil (e.g., via a feedback loop) to form a distortion field-map (e.g., indicative of an optimal position of the shim coil).

Further described are controllers configured to operate the motorized robot system or the MR-compatible robot systems to perform any of the methods described herein.

Various aspects also include a non-transitory computer readable medium of the controllers discussed herein.

Systems, methods, and devices are disclosed for a magnetic resonance compatible (“MR compatible”) robotic cantilever platform that can perform activities inside an MRI scanner bore under high magnetic fields. The 3-degree-of-freedom cartesian platform is MRI-safe and is configured to handle a heavy-weight payload. It is also customizable and scalable (via a parametric design).

The three degrees of freedom (x, y, z) motorized platform can position a shim coil, a coil intended to improve magnetic field homogeneity in cases where there are objects on or inside a subject that reduce the field homogeneity to levels that inhibit acquiring good quality MRI images, at a desired location above a subject using a cantilevered arm. The motorized platform can be fabricated with three plastic linear lead screws that permit decoupled translation in the head/foot, anterior/posterior, and right/left directions. The linear lead screws can be driven by a powerful and precise MR-compatible pneumatic actuator and encoder system. The control electronics for the motorized platform, such as pneumatic valves, power supply, and valve controllers, can be placed in the MRI control room. All components will be non-metallic and MRI-compatible to ensure image quality and MRI safety. The motorized arm can be mounted distally onto the scanner's bed rail or mounted to a table placed at the rear of the scanner.

According to one aspect, the present disclosure is directed to an MR-compatible robot system comprising: an MR-compatible beam; an MR-compatible robot platform coupled to the beam; and an MR-compatible motor configured to move the beam relative to the platform.

In one embodiment, the MR-compatible motor includes a pneumatic, hydraulic, or piezoelectric motor.

In one embodiment, the MR-compatible motor includes a quadrature encoder.

In one embodiment, the controller is configured to position the beam relative to the platform with 2 mm positional accuracy.

In one embodiment, the system further includes a payload coupled to the beam.

In one embodiment, the beam is a cantilever beam.

In one embodiment, the beam includes fiberglass.

In one embodiment, the platform and the MR-compatible motor are configured to move the beam in two degrees of freedom.

In one embodiment, the platform and the MR-compatible motor are configured to move the beam in three degrees of freedom.

In one embodiment, the platform includes a plurality of MR-compatible motors and the plurality of MR-compatible motors are configured to control both the position and orientation of the beam relative to the platform.

In one embodiment, the platform includes a x-stage lead screw, a y-stage lead screw, and a z-stage lead screw configured to control the position and/or orientation of the beam.

In one embodiment, the platform is configured to control the beam with a range of motion greater than 2 cm, such as greater than 3 cm, greater than 4 cm, greater than 5 cm, greater than 6 cm, greater than 7 cm, greater than 8 cm, greater than 9 cm, greater than 10 cm, greater than 11 cm, greater than 12 cm, greater than 14 cm, greater than 16 cm, greater than 18 cm, greater than 20 cm, greater than 25 cm, or greater than 30 cm.

In one embodiment, the platform includes an MR-compatible material.

In one embodiment, the MR-compatible materials include at least one of:

PLA, Nylon, fiberglass, Acrylic and/or other plastics. In some examples, the MR-compatible material includes a 3D-printable material.

It should be understood that the examples described herein are only non-limiting examples, and that embodiments of the present disclosure can be used for a variety of measurement techniques.

Additional advantages of the disclosed systems and methods will be set forth in part in the description which follows and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

### BRIEF DESCRIPTION OF THE DRAWINGS

Example features and implementations are disclosed in the accompanying drawings. However, the present disclosure is not limited to the precise arrangements and instrumentalities shown.

FIG. 1 shows an exemplary motorized robotic system having a cantilever beam to extend into the available clearance space of the bore of an MRI scanner when the patient is inside to position interventional and scanning assisted instruments during an MRI scan, according to one implementation.

FIGS. 2A, 2B, and 2C respectively show a perspective (FIG. 2A) and orthographic projections of top (FIG. 2B), and side (FIG. 2C) views of the exemplary motorized robotic system of FIG. 1.

FIG. 2D shows a perspective view of the x-stage sub-assembly of the motorized robotic system of FIG. 1.

FIG. 2E shows a perspective view of the y-stage sub-assembly of the exemplary motorized robotic system of FIG. 1.

FIG. 2F shows a bottom view of a sliding block of the y-stage sub-assembly of FIG. 2E.

FIGS. 2G and 2H, respectively, show a perspective and side views of a z-stage sub-assembly of the exemplary motorized robotic system of FIG. 1.

FIG. 2I shows a side view of a sensorimotor unit of a motorized robotic system according to one implementation.

FIG. 2J shows the exemplary system motion of a motorized robotic system according to one implementation.

FIG. 2K shows a force diagram of an example motorized robotic system for mechanical modeling. Forces pointing towards a rod are forces applied to the rod, and forces pointing out from the rod are forces applied by the rod to the sliding block. F_{y1} and F_{y2} are obtained by the equilibrium of the Y-stage sliding block. Fₓ₁ and Fₓ₂ are obtained by the equilibrium of the system with the X-stage sliding block and the entire Y-stage assembly. Note that the coordinate for the X-stage guiding rod starts on the right end for ease of illustration, which renders the direction of the positive moment opposite to that of other stages.

FIGS. 3A and 3B each show another exemplary motorized robotic system in an MRI scanner with a bottom-loaded counterweight according to another implementation.

FIG. 4A shows an example controller for the exemplary motorized robotic system according to one implementation.

FIG. 4B shows another view of the optical encoder of FIG. 4A where a pair of transmitters and receivers separated by an extrusion member is used to measure a rotational position of a lead screw.

FIG. 4C an example of a graphical user interface (GUI) according to one implementation. The GUI can be used to control the movement of the robot in addition to settings of the instrument. The GUI shows the driver of the cantilevered robot on top and shim current passing through the shim coil below (the lower picture is a digital oscilloscope).

FIG. 4D shows an example of a software feedback loop for the safe control of the motion of the motorized robotic system according to one implementation.

FIGS. 5A and 5B show flowcharts depicting exemplary methods of using the motorized robotic systems described herein for interventional (FIG. 5A) and diagnostic (FIG. 5B) procedures.

FIGS. 6 and 7 shows an example prototype motorized robotic system developed in a study to image patients with implanted ICD. Specifically, FIG. 6 shows (Panels A-D) Images from a patient with an implanted ICD. (Panel A) Radial UTE MR image with target ROI region (white square) and void (black) region (arrows) indicating a region of largest ICD generated field inhomogeneity. (Panel B) simulated field map of a dipole fitted to the UTE field map, with dark colors indicating regions of largest inhomogeneity (Blue: positive (higher) field, red: Negative (lower) field. Field contours lines (black) are at 25 parts per million (ppm) of the static magnetic field increments). (Panel C) 2D coronal current pattern of the shim coil, with color indicating current polarity). (Panel D) Simulated total field after shim-coil correction. Note far smaller changes in a box. Views of shim coil (Panel E) interior form (1/2) and (Panel F) fully assembled exterior. The rectangular coil is oriented with its long axis along the scanner's superior-inferior (z) and its short axis along the left-right (x) directions. It produces a magnetic field along the anterior-posterior (y) direction.

FIG. 7 shows a motorized cantilevered system to remotely position the shim coil in the MRI. (Panel A) CAD model of the entire system. (Panel B) CAD focusing on cantilevered arm and motors. (Panel C) View from the rear of MRI bore, showing pneumatic motors and back end of a cantilevered beam, with counterweights balancing the moment arm produced by the shim coil. (Panel D) view from front of bore, showing a subject inside the bore with a cardiac coil above the abdomen with the shim coil above, suspended from the distal end of the beam.

FIGS. 8A-8F shows scans of patients with implanted ICD (and test model) acquired in the study. Specifically, FIG. 8A shows (Panels A-F) Displacing the ICD case increasingly lower along the SI direction on the swine chest caused an increase in the extent of the non-uniformity artifact (black void), and additional signal-loss changes (yellow arrows). FIG. 8B shows navigated 3D LGE in sagittal and axial direction; prior to (Panels A, C) and after (Panels B, D) shim correction. Red arrows indicate location of artifacts. Note complete axial image in Panel D. FIG. 8C shows interactive shimming in a phantom with an overlaid ICD, which causes an image void. (Panel A) Changing the shim current with continuous axial GRE visualization, leading to a smaller void. (Panel B) changing the shim current with continuous sagittal SSFP visualization. Note the increasingly larger distances between the SSFP bands. The white square indicates the region corrected. FIG. 8D shows Sagittal SSFP of swine with ICD. Showing changes at applied shim currents of (Panel A) 0, (Panel B) 2.5, and (Panel C) 3.5 Amperes. White dashed square indicates region where homogeneity progressively improved. FIG. 8E shows changes during changes in shim coil positioning with cine imaging. (Panel A) The state before the ICD was added (which serves as the baseline for restored image quality). (Panel B) worst post-ICD state, (Panel C) slightly improved post-ICD state, (Panel D) increasingly improved post-ICD state, (Panel E) most improved post-ICD state. Note that LV visualization in Panel E is almost as good as in Panel A. Red arrows point to inferior border of void which is continuously moved between Panels B and E. FIG. 8F shows a comparison of 2D LGE (Panel A) prior to shimming and (Panel B) after shimming using the CSS system. The image prior to shimming has severe distortions at the top of the Left ventricle, whereas the image after shimming allows visualizing the entire LV.

### DETAILED SPECIFICATION

The details of one or more embodiments of the invention are set forth in the accompanying drawings and the description below. Other features, objects, and advantages of the invention will be apparent from the description and drawings and from the claims.

Throughout the description and claims of this specification, the word “comprise” and other forms of the word, such as “comprising” and “comprises,” means including but not limited to, and is not intended to exclude, for example, other additives, components, integers, or steps.

To facilitate an understanding of the principles and features of various embodiments of the present invention, they are explained hereinafter with reference to their implementation in illustrative embodiments.

### Example Motorized Robotic System

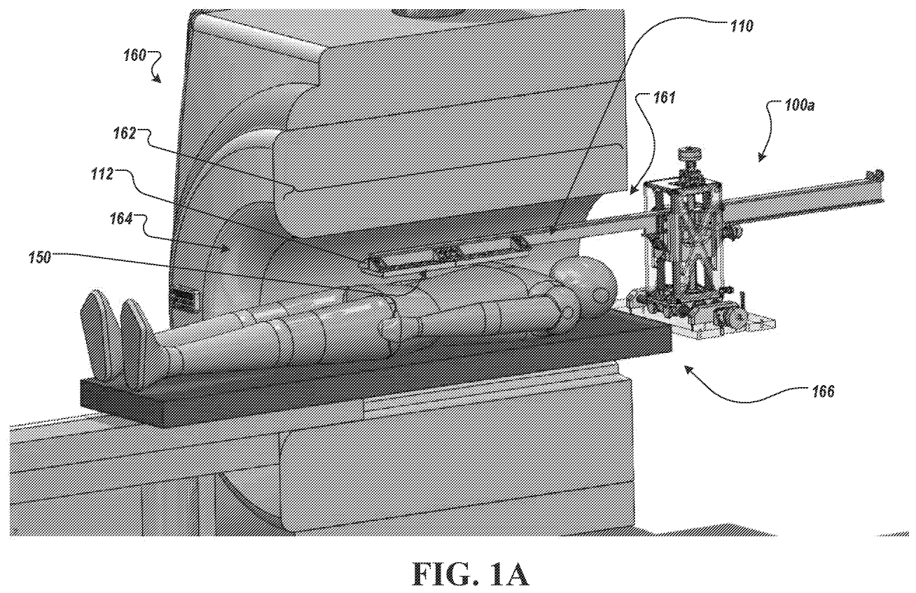

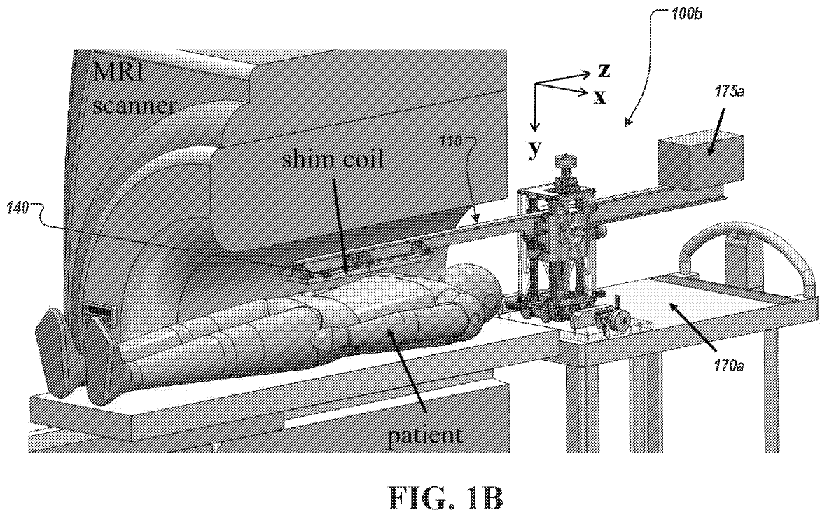

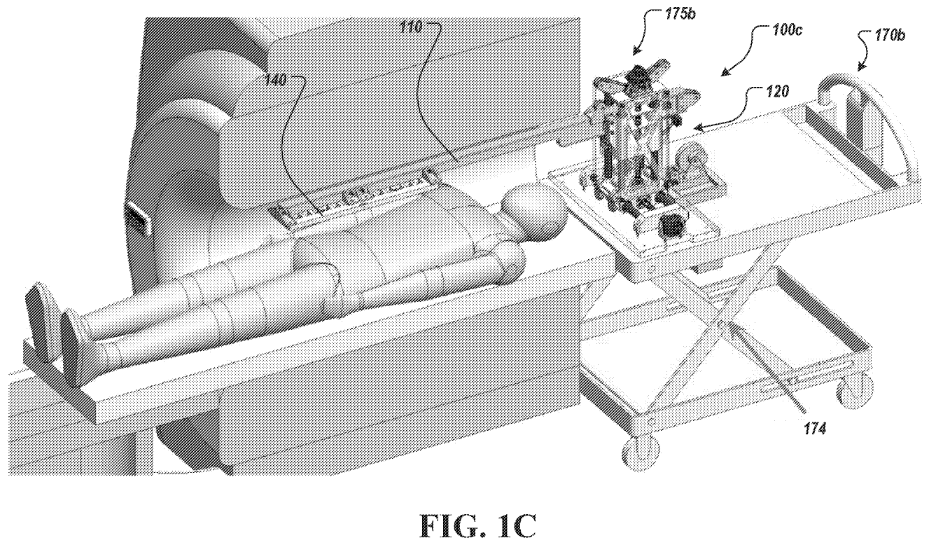

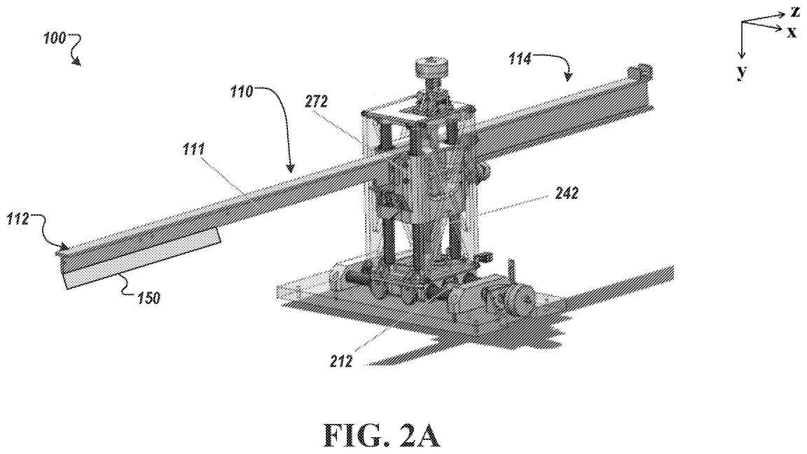

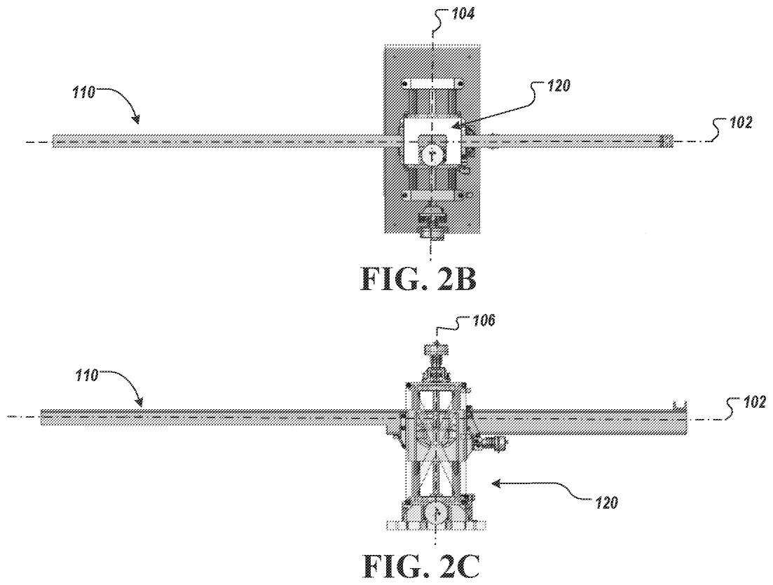

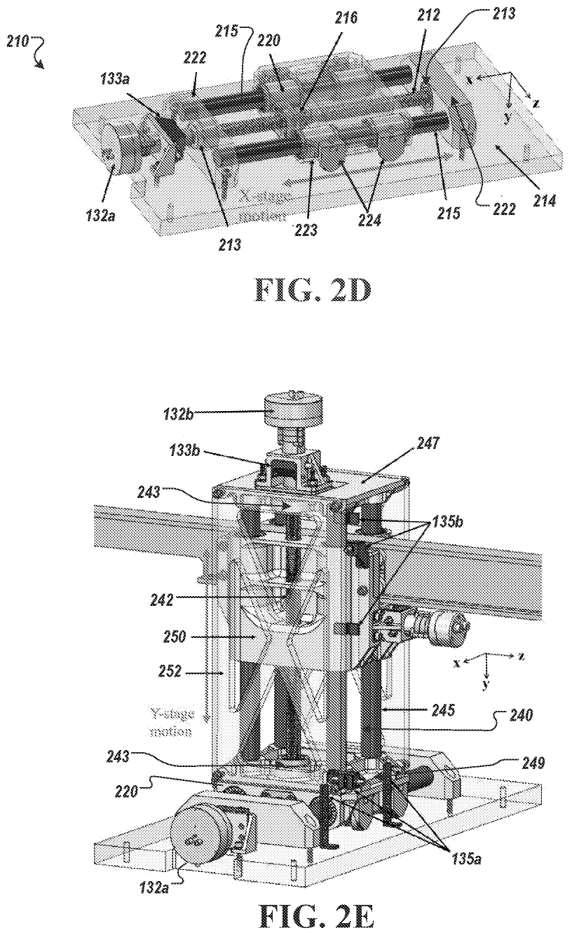

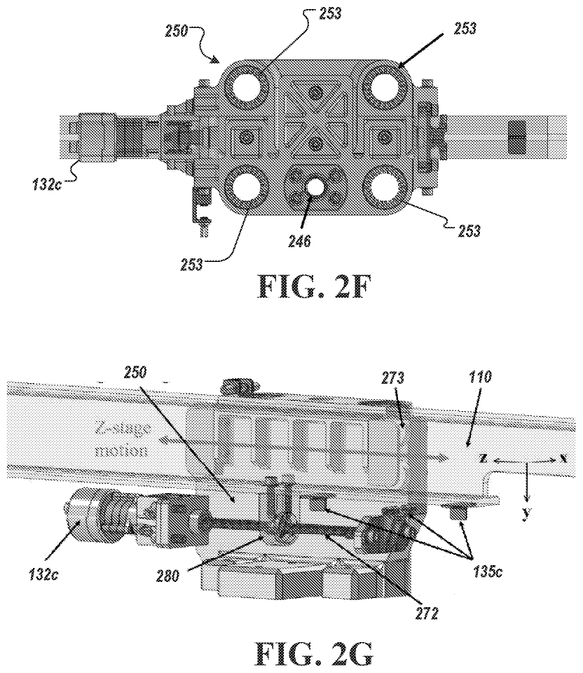

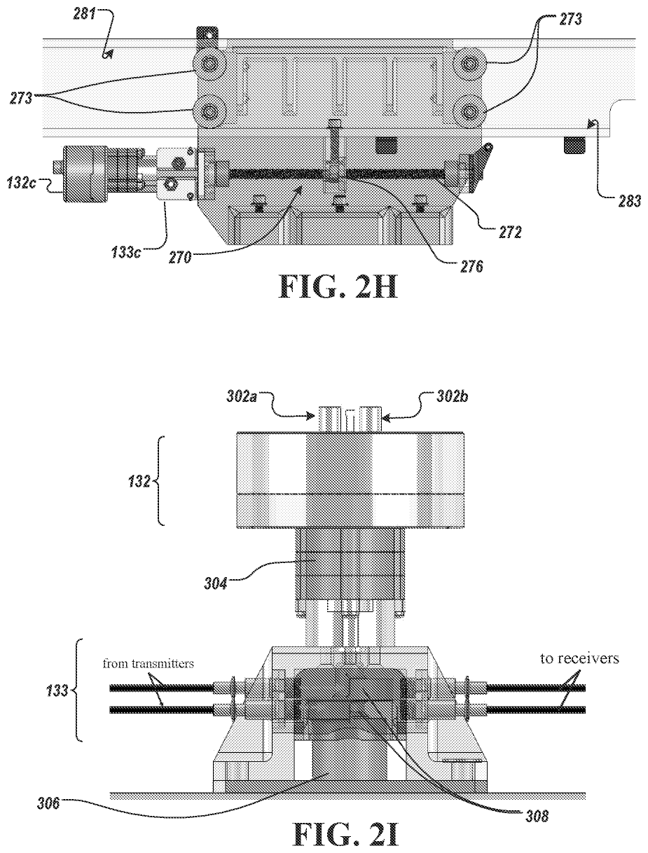

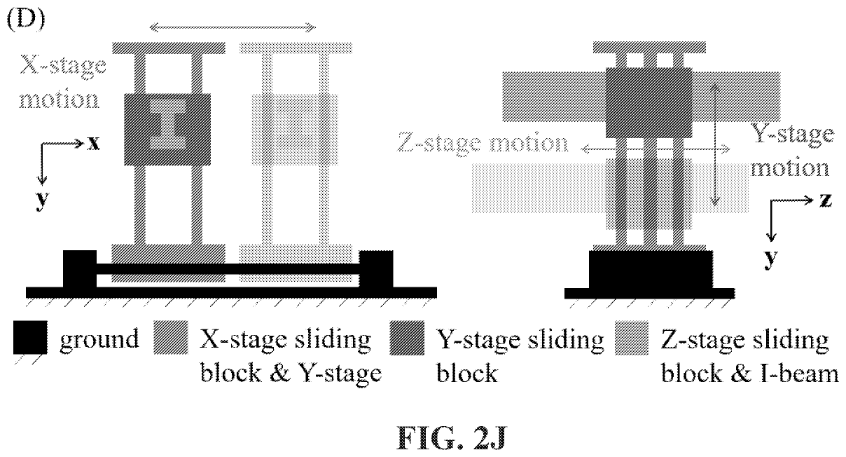

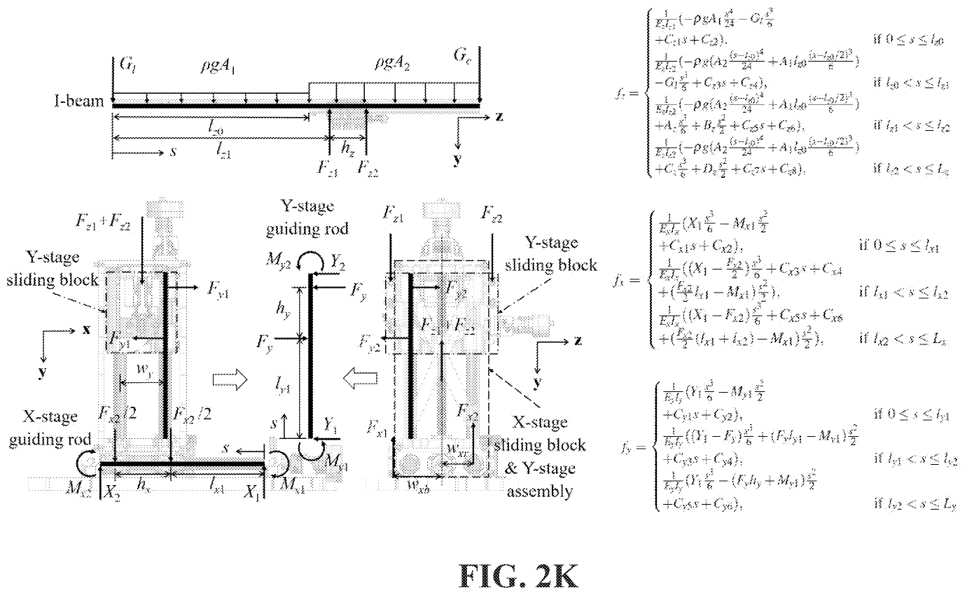

An example embodiment of the present disclosure includes a motorized robotic system 100, such as the one shown in FIGS. 1A-2I.

FIGS. 1A-1C show an example motorized robotic system 100 (100a, 100b, 100c) disposed in an MRI scanner 160 according to one aspect of the present disclosure. The motorized robotic system 100 is positioned at a proximal end 166 of the MRI scanner 160. The term “proximal end,” as used herein, refers to the side of the MRI scanner that is nearer the motorized robotic system during use, and the term “distal end”, as used herein, refers to the side of the MRI scanner member that is away from the motorized robotic system during use. Thus, the “proximal end” and “distal end” relate to the position of the motorized robotic system relative to the MRI scanner. Although the motorized robotic system 100 shown in FIGS. 1A-1C are depicted such that the proximal end is towards the head of a patient, the motorized robotic system may also be positioned at or about the feet of the patient. The arm member 110 of the motorized robotic system 100 is disposed through an opening 161 of the MRI scanner 160 into a bore or elongated section 162 such that an instrument 140 at a first side 112 of the arm member 110 can be placed above a patient.

Referring specifically to FIGS. 1B-1C, the motorized robotic systems 100b, 100c can be engageably coupled with a counterweight system 175 (175a, 175b).

Counterweight system 175a includes a load-based counterweight system wherein a weight is disposed on the arm member 110 at a location opposite the instrument 140. Counterweight system 175b is a pulley-based counterweight system that uses a series of pulleys to generate a self-balancing mechanism. The counterweight system can provide adaptive stabilization in order to balance the arm member upon movement of the robotic system. The motorized robotic systems 100b, 100c shown in FIGS. 1B-1C are disposed on an adjustable chassis 170a, 170b in order to enable rapid movement of the motorized robotic system 100b, 100c in and out of the scanning room. The adjustable chassis 170b shown in FIG. 1C further includes a y-stage adjustment mechanism 174 which allows an operator to raise and lower the position of the motorized robotic system 100c to an appropriate height with respect to the MRI scanner.

The motorized robotic system can be adapted with various types of instruments. In some aspects, the instrument includes a shim coil configured to generate a magnetic field to create a controlled magnetic field, the shim coil defining an electromagnet secured (e.g., fixedly or movably) to the first end of the arm member. In some aspects, the instrument includes a second robotic assembly coupled to an ablation instrument (RF or thermal), an ultrasound probe (e.g., of the liver), a biopsy excising instrument, or a fluoroscopy instrument (or another interventional instrument). In some aspects, the instrument includes an ablation instrument (RF or thermal), an ultrasound probe (e.g., of the liver), a biopsy excising instrument, or a fluoroscopy instrument (or another interventional instrument).

Referring now to FIGS. 2A-2I, the motorized robotic system 100 includes an arm member 110 comprising an elongated beam 111 (e.g., a cantilever beam) that extends along a longitudinal axis 102. The arm member 110 has a first side 112 and a second side 114 away from the first side 112. The first side 112 of the arm member 110 includes an instrument 140 (e.g., coil, ablation instrument, surgical device) that is configured to be at least partly positionable within a bore or elongated section 162 of an MRI scanner 160. To withstand applied forces, the arm member can formed using an MR-compatible material having high strength and stiffness, such as fiberglass-reinforced-polyester I-beam for its high strength and bending stiffness. This cantilever-like structure allows the base assembly 120 of the motorized robotic system 100 to be placed outside the MRI scanner bore or elongated section 162 while supporting the payload remotely, minimizing the space occupied inside the bore or elongated section 162. The arm member 110 shown in FIGS. 2A-2I includes a partial cut, which further reduces the amount of occupying space with the bore or elongated section 162. The bore or elongated section 162 has a distal end 164 and a proximal end 166, and the MRI scanner 160 has an opening 161 of the bore or elongated section 162 at the proximal end 166. In some aspects, the arm member and base assembly are substantially free of ferromagnetic materials. In some aspects, the arm member and base assembly are substantially free of paramagnetic materials. In some aspects, the arm member and base assembly are substantially free of ferromagnetic and paramagnetic materials. For example, the arm member and base assembly can comprise an MR-compatible material (e.g., selected from the group consisting of Polylactic acid (PLA), nylon, fiberglass, and acrylic).

The motorized robotic system 100 also includes a base assembly 120 that is moveably coupled to the arm member 110 and positioned towards the second side 114 of the arm member 110. As shown in FIGS. 2A-2I, the arm member 110 is disposed within a housing 122 of the base assembly 120 such that it may be moved in a horizontal axis 104, a vertical axis 106, and/or a longitudinal axis 102 (illustrated as x, y, and z, respectively in FIG. 2A).

The base assembly 120 has a motion unit 130 including one or more motors 132a, 132b, 132c that are configured to, while a patient is located in the bore or elongated section 162, (i) move the arm member 110 along the longitudinal axis 102 from (a) a first position having the instrument 140 outside of the bore or elongated section 162 to (b) a second position within the bore or elongated section 162 to place the instrument 140 at or toward the distal end 164 of the bore or elongated section 162 and (ii) move the arm member 110 along the horizontal axis 104 and/or the vertical axis 106 perpendicular to the longitudinal axis 102 at the second position to position the instrument 140 at or around the distal end 164 of the bore or elongated section 162 in relation to the patient.

In some aspects, the motorized robotic system includes a counterweight system coupled to the arm member at or proximal to the second end. The counterweight system can be configured to be dynamically translatable along the longitudinal axis and/or the vertical axis to account for the movement of the arm member (e.g., longitudinal translation).

In some aspects, the motion unit includes one or more lead screws operatively coupled to the one or more motors such that actuation of the one or more motors causes the lead screw to rotate such that the arm member translates relative to the base assembly.

In some aspects, the motion unit includes a first lead screw positioned along the longitudinal axis, a second lead screw positioned along the horizontal axis, and a third lead screw positioned along the vertical axis.

FIGS. 2D-2G illustrates examples of various mechanisms that can be used in the motion unit 130 to move the arm member 110 relative to a patient in the MRI scanner 160.

FIG. 2D shows an x-stage assembly 210 of the motion unit 130 that can be used to move the arm member 110 along the horizontal axis 104 (e.g., in the x-direction). The x-stage assembly 210 is provided on a base plate 214 for support. The base plate can include a structurally rigid surface comprising an MR-compatible material (e.g., a 1″ acrylic surface) that does not interfere with image recordings of the MRI scanner. Although not shown in FIG. 2D, it may be advantageous for the base plate 214 to be disposed on or secured to a portable chassis, such as the adjustable carrier 170 of FIGS. 1B-1C so that operators may rapidly move and/or deploy the motorized robotic system to a position proximate to the MRI scanner.

Attached to the base plate 214 is a motor 132a that is operatively coupled to a first lead screw 212 and an encoder 133a. The first lead screw 212 shown in FIG. 2D comprises a threaded rod (e.g., nylon with ¾″-10 thread) that is used to drive a hex nut 216 embedded in a first sliding block 220. When the first lead screw 212 is radially rotated by the motor 132a, the threadings on the first lead screw 212 engage with threadings in the nut 216 such that the first sliding block 222 moves about the horizontal axis 104 (in the x-direction). The nut 216 can be made of an MR-compatible material, such as nylon. The first lead screw 212 is supported on both ends by support blocks 222 via rolling elements 213 (e.g., radial deep groove ball bearings). The term “rolling elements” as used herein, broadly refers to rotating connectors having friction-reducing elements. By way of non-limiting example, the rolling elements 213 can include deep groove ball bearings that are made of plastic rings and glass balls or any other MR-compatible material. Two guiding rods 215 are symmetrically placed on both sides of the first lead screw 212 to provide structural stability in view of applied forces to the system. While the size and positioning of the guiding rods on the base plate may be varied, the guiding rods can, in some examples, have a 1″ outer diameter. In some examples, the guiding rods include an MR-compatible material. For example, the guiding rods can be made of molybdenum disulfide (MDS) filled nylon that is self-lubricating and has low friction surfaces which provide facilitated lateral motion of the sliding block about the x-axis. The guiding rods 215 shown in FIG. 2D are coupled with linear sliding elements 223 (e.g., linear slide bearings) to form a connection point with the first sliding block 220. The first sliding block 220 is further supported on the base plate 214 by ball bearings 224 that can act as wheels to partially bear the weight of the upper structures of the motorized robotic system 100. Although the support blocks 222 and the sliding block 220 shown in FIG. 2D were custom-designed and fabricated using 3D printing with polylactic acid (PLA), other materials and fabrication techniques may also be used.

Referring now to FIG. 2E-2F, an example of a y-stage assembly 240 of the motion unit 130 is illustrated. The y-stage assembly 240 includes a second lead screw 242 that can utilize a similar structure and configuration of nut, guiding rod, and sliding elements as the x-stage assembly 210. The second lead screw 242 is offset from the center to make room for a third lead screw that extends in the z-direction. Guiding rods 245 are used to provide balanced support for a second sliding block 250 that is configured to translate along the vertical direction (i.e., along the y-axis). The second sliding block 250 can, in various examples, be formed using two separate pieces for ease of assembly. As shown in FIG. 2F, the second sliding block 250 includes several sliding elements 253 (e.g., linear slide bearings) that are configured to allow the second sliding block 250 to move vertically about the guiding rods 245 with minimal friction. The second sliding block 250 further includes a nut 246 that causes the second sliding block 250 to move in the y-direction upon rotation of the second lead screw 242 by the motor 132b. The second lead screw 242 is supported by rolling elements 243 above and below the second lead screw 242. In some aspects, it is advantageous to use an axial rolling element (e.g., axial ball bearing) from below and a radial rolling element (e.g., radial ball bearing) from above since a portion of the second lead screw below the nut can be vertically compressed by the weight of a z-stage assembly and other parts of the motorized robotic system. The y-stage guiding rods 245 are constrained within a housing 252 of the base assembly 120 and are connected to each other by an upper support plate 247 and a lower support plate 249 of the housing 252. The lower support plate 249 is securely fixed to the first sliding block 220 of the x-stage assembly 210.

Referring now to FIGS. 2G-2H, a z-stage assembly 270 is shown according to one aspect of the present disclosure. A third lead screw 272 comprising a threaded rod is nested in the second sliding block 250, which is configured to be translatably moved in the y-direction. The third lead screw 272 shown in FIG. 2G includes an MR-compatible material, such as a nylon threaded rod, that is supported on both ends by rolling elements 273 (e.g., ball bearings). The z-stage assembly 270 includes a third sliding block 280 secured to the arm member 110. The third sliding block 280 is engageably coupled with the third lead screw 272 via a nut such that the rotation of the third lead screw causes the third sliding block 280 and thus the arm member 110 to move along the longitudinal axis 102 (e.g., the z-direction). To make the third lead screw 272 assembly compact and reduce the weight, the geometry of the arm member can be utilized instead of using a guiding rod. For example, FIG. 2G shows rolling elements 273 (e.g., ball bearings or wheels) that are secured to the second sliding block 250 and are engageably coupled to the top 281 and a bottom 283 of the I-beam flanges of the arm member 110 in a vertical direction. In this regard, the arm member 110 can be secured in the y-direction by the rolling elements 273 and in the x-direction by the second sliding block, hence only permitting translation in the z-direction. To ensure the motorized robotic system does not interfere with image recordings of the MRI scanner, it is further advantageous to use fasteners comprising MR-compatible materials, such as nylon.

FIG. 2J shows a diagram of the Cartesian-based motion of the exemplary system. An example force diagram of a motorized robotic system is shown in FIG. 2K (further description of the mechanical modeling is included in U.S. Provisional Patent Application No. 63/496,580, which is incorporated by reference in its entirety). Because the motorized robotic system operates in a small environment within an MRI scanner, ensuring both mechanical compliance and precise manipulation carries several obstacles. Thus, the mechanical model can be used to assist with geometric and material selection for forming a motorized robotic system configured to operate within the scanner.

### Example Sensorimotor Unit

FIG. 2I illustrates an example of an individual sensorimotor unit 300 that can be used to drive rotational movement of any of the lead screws of the x-stage assembly 210, y-stage assembly 240, or z-stage assembly 270. The sensorimotor unit 300 includes a motor 132 (e.g., 132a, 132b, 132c) and an encoder assembly 133 (e.g., 133a, 133b, 133c). The motor 132 includes various components such as a stator, cap, and a rotor to convert an MR-compatible actuating input (e.g., pneumatic or hydraulic) into rotational energy. Although the motor 132 depicted in FIG. 2I is formed with a 3D-printed resin fastened using plastic hardware (e.g., screws, all-thread rods, and hex nuts made of nylon), other MR-compatible materials may also be used. The motor 132 includes two inlets 302a, 302b configured to receive an actuating input through an MR-compatible tube (e.g., polyurethane (PU)) in order to enable bi-directional rotation. The motor further includes a gearbox 304 (e.g., a planetary gearbox) formed of an MR-compatible material (e.g., plastic) that can modify the angular speed and/or torque of the motor 132. The motor is further coupled with an optical quadrature encoder 133 configured to measure an output shaft rotation. Two transmitter-receiver channels are connected to the motor mounting block for optical signal encoding. The encoder assembly 133 uses two adjacent extrusions 308 on the coupling 306 that connects the motor output shaft to a corresponding lead screw. Each extrusion 308 blocks the corresponding transmitter-receiver channel for half a rotation, generating a typical quadrature signal. The light of the encoders is transmitted by optical fibers with polymer cores and jackets. The encoder assembly 133 is entirely enclosed to prevent the disturbance by ambient light. Each stage assembly (210, 240, 270) of the motion unit 130 can also be equipped with an optical limit switch (135a, 135b, 135c) which includes one transmitter-receiver channel and detects when the sliding block (moves to the limit position. Because the motor 132 is driven with an MR-compatible actuating input (e.g., pneumatic or hydraulic), long tubes and optical fibers can be used to allow remote control of the motorized robotic system from outside the scanning room.

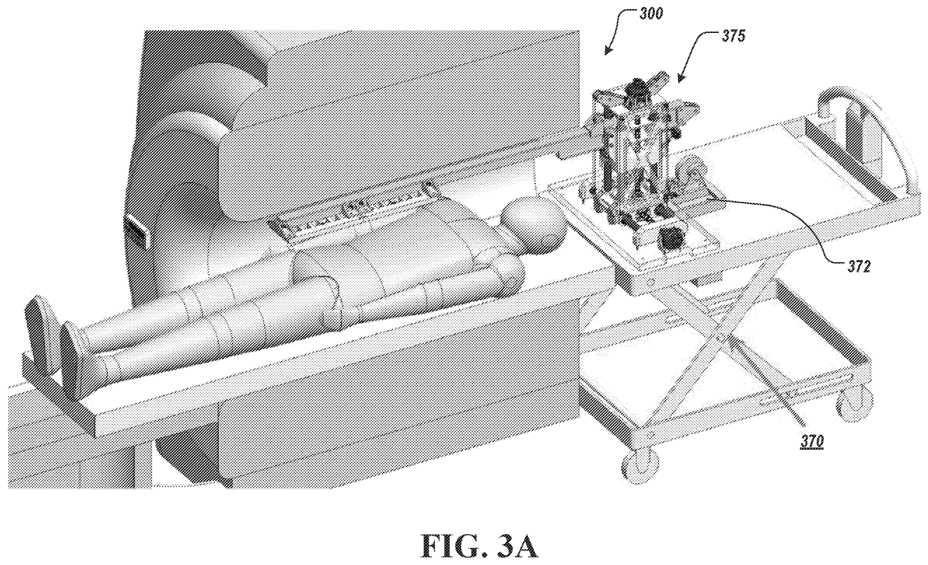

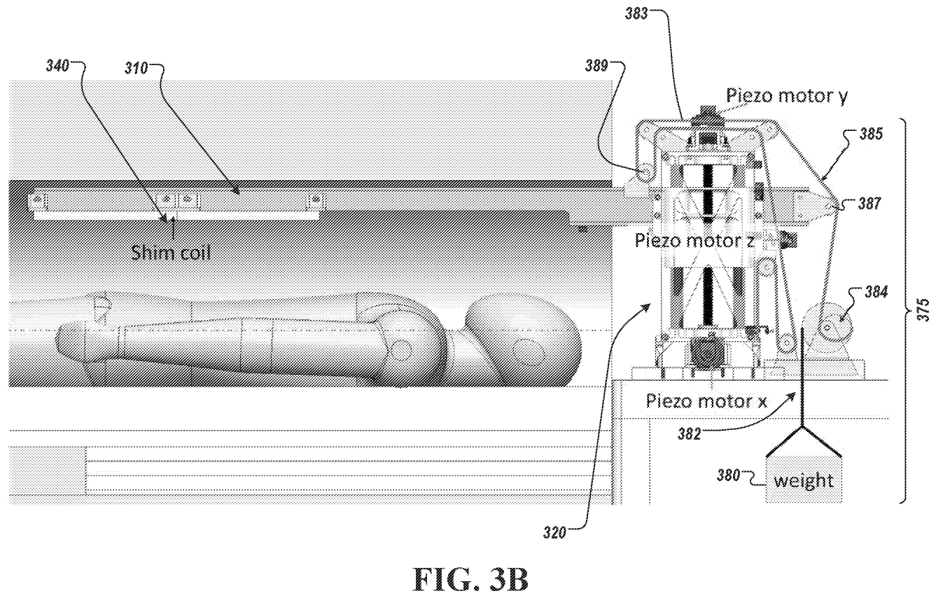

FIGS. 3A-3B shows a motorized robotic system 300 according to another implementation. The motorized robotic system 300 is positioned at a proximal end of the MRI scanner. The motorized robotic system 300 includes an arm member 310 comprising an elongated beam (e.g., a cantilever beam) and a base assembly 320 moveably coupled to the arm member. Disposed on the arm member is an instrument 340 (e.g., shim coil) that is configured to be at least partly positionable within a bore or elongated section of an MRI scanner. The arm member 310 of the motorized robotic system 300 is disposed through an opening of the MRI scanner into a bore or elongated section such that an instrument 340 can be placed above a patient.

As illustrated in FIG. 3A, the motorized robotic system 300 is disposed on an adjustable carrier 370 that is configured to be maneuverable (e.g., using hydraulic or pneumatic actuation) about the horizontal and/or vertical axes to move the base assembly about a height and/or a center of the bore or elongated section. The motorized robotic system 300 further includes a counterweight system 373. The counterweight system 375 shown in FIGS. 3A-3B includes a balancing weight 380 secured to the arm member 310 via a pulley system 382 including one or more pulleys. As used herein, the term “pulley” refers to a rotatable wheel along which a cable may move. Thus, the term “pulley system” can be used to refer to a collection of pulleys and their supporting elements (e.g., cable(s), fasteners) used to raise or lower the balancing weight. The pulley system 382 includes a cable 383 that engages with a cam wheel 384 and contacts the arm member 310 to form a tendon 385 located at a side of the arm member 310 away from the instrument 340. In various aspects, the cam wheel can be articulated (e.g., using a controller) to adaptively raise and lower the balancing weight. The pulley system 382 engages with the arm member 310 via a second pulley 389 disposed between the first pulley 387 and the instrument 340. The pulley system 382 is arranged about the housing 320 such that the balancing weight 380 directs a tensile force on the arm member 310 at the tendon 385 to adaptively stabilize the motorized robotic system 300 as the arm member 310 is translated along the longitudinal axis. The balancing weight 380 is connected to the pulley system 382 through an opening 372 in the adjustable carrier 370. When the arm member 310 moves longitudinally, the wire-wrapping angle of the cam wheel 384 changes, and hence the moment arm of the tendon 385 tension force changes. Since the moment on the cam wheel 384 is held constant by the balancing weight 380, the tendon 385 tension self-adjusts according to the arm member's 310 motion in a z-direction. The arm member's 310 y-direction (vertical) motion is decoupled from the cam wheel 384 motion by the pulley system 382 that compensates the change in the tendon 384 length caused by the y-direction motion. The counterweight system 375 is mounted on the base assembly 320 (e.g., either to the y-stage assembly or an x-direction guiding rail) that is fixed to the adjustable carrier 370 and moves with the x-stage assembly, hence the mechanism can also be decoupled from the x-direction motion. The counterweight system 375 is capable of partly balancing the moment generated by the payload at any robot configuration, while also removing bulky counterweight to reduce the space occupation.

Another example of a counterweight system is shown in FIG. 1B. The counterweight system 175b is coupled to the arm member 110 at a position away from the instrument 140. In some aspects, the counterweight system 375 is configured to be dynamically translatable (e.g., using rolling elements) along the longitudinal axis (z-direction) in response to the longitudinal movement of the arm member 310.

### Example Controller

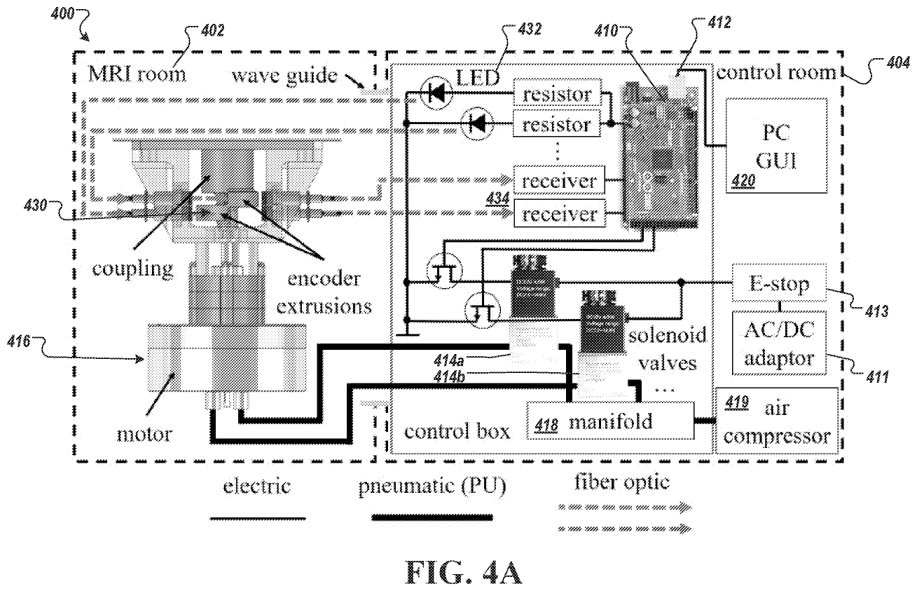

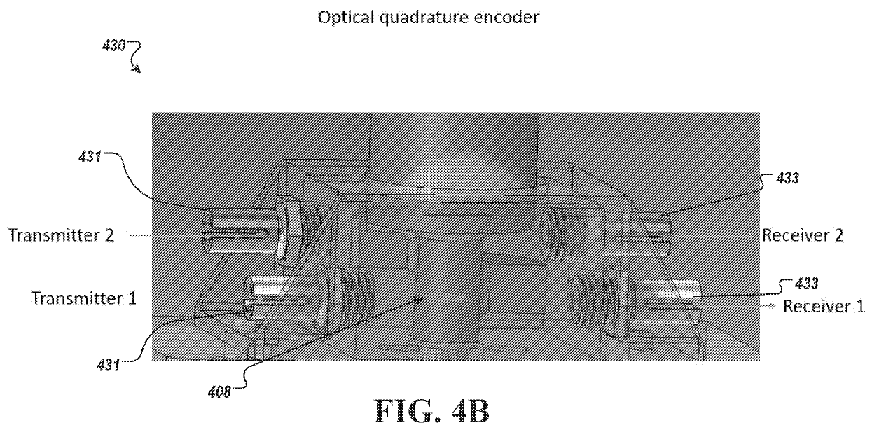

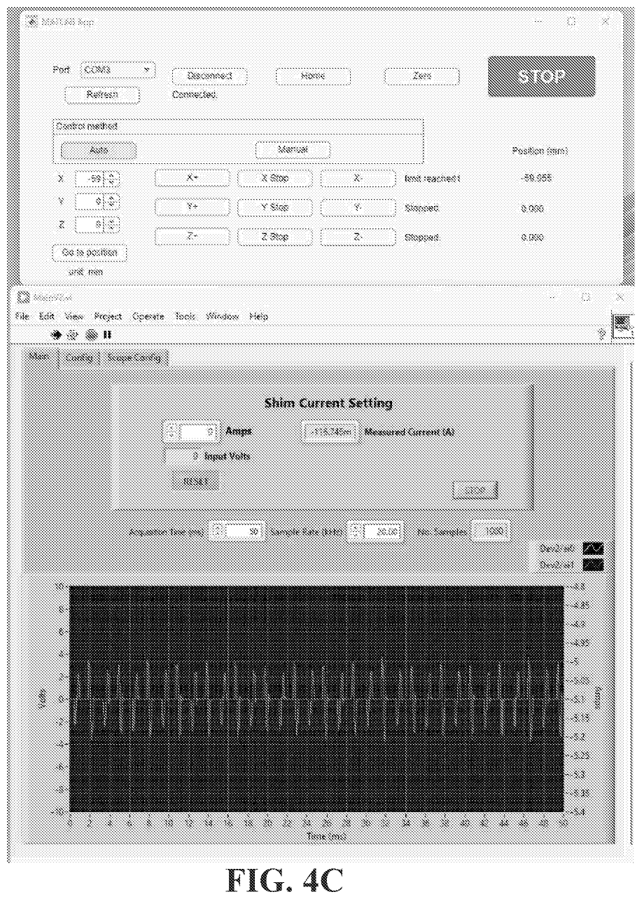

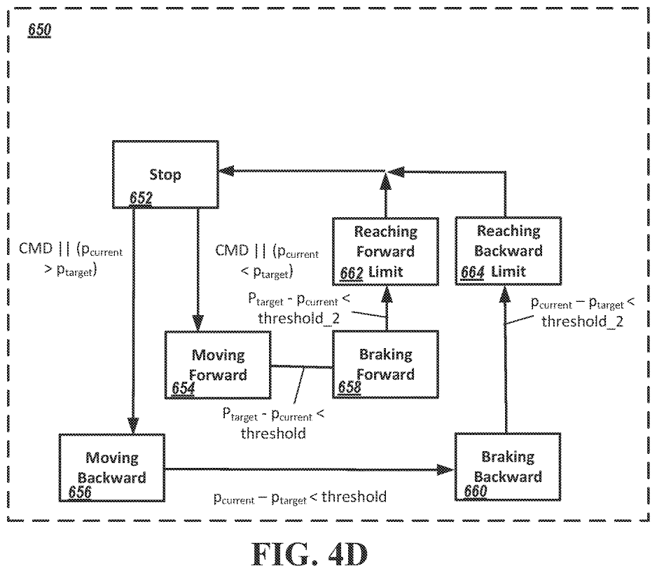

FIG. 4A illustrates an example robot control system 400 for the exemplary MR-compatible robot system comprising feedback loops for robotic actuation. The robot control system 400 can be designed such that the ferromagnetic components can be placed outside the MR room 402. Thus, an MRI operator can control the robot remotely from the MRI control room 404 on a computer through a graphical user interface (GUI) 420. In various implementations, the GUI 420 is designed using a Matlab APP Designer. The GUI 420 can communicate with a controller 410 (e.g., Arduino Mega 2560) through a serial port 412, sending commands and reading robot status information. Two normally closed solenoid valves 414a, 414b (2 position 2 port, TAILONZ Pneumatic, China) can be used to control each motor 416, and each solenoid valve is controlled by the controller 410 through an N-channel MOSFET. The inlet tubes of solenoid valves can be merged using a pneumatic manifold 418 and connected to a portable air compressor 419. The valves 414a, 414b can then be connected to the power supply 411 via an emergency-stop switch 413 which allows the technologist to quickly cut off the power in emergency situations. For the optical encoders 430, LEDs 432 are used as the transmitter light source, and receiver PCBs 434 with photodetectors (Industrial Fiber Optics, Inc.) can be used to convert light signals into digital signals which are then processed by the controller 410. FIG. 4B shows another view of the optical encoder 430 where a pair of transmitters 431 and receivers 431 separated by an extrusion member 408 is used to measure a rotational position. In examples where the valves cannot be proportionally actuated, it is possible to employ a bangbang controller for motor control. The switching points of the controller can be carefully tuned to minimize overshooting.

In various aspects, the motorized robotic system further includes a controller. In various examples, the controller can be configured, via software, to detect an impact or contact of the instrument or arm member to a wall of the bore or elongated section, to the patient, or an object in the bore or elongated section. In various aspects, the base assembly, the arm member, and/or the instrument include one or more sensors (e.g., a quadrature encoder) that provide spatial data to the controller associated with a current position of the arm member or the instrument. Based on the spatial data, the controller can be configured to halt movement of the system based on the detection. In various aspects, the controller is further configured to: receive the spatial data from the one or more sensors; and control the one or more motors of the motion unit to move the arm member from the current position to a target position.

In some aspects, the controller is configured to: receive movement data associated with the patient; and control the one or more motors to move the arm member based on the patient movement data (e.g., to account for breathing).

In various implementations, the motorized robotic system can use a movement control platform. By way of non-limiting example, the movement control platform for the robotic system can include a MATLAB application on PC and an Arduino code. In some implementations, the control software runs a graphical user interface (GUI) and a 20 Hz timer that reads the user instruction and feedback from the sensors and sends command to the Arduino via serial communication in each cycle. In an example, shown in FIG. 4D, the movement control platform 650 can maintain a finite state machine, such that each robot axis is in one of the seven states: stopped 652, moving forward 654, moving backward 656, braking forward 658, braking backward 660, and reaching forward 662, or reaching backward limit 664. In each controller cycle (which has a much higher frequency than the MATLAB timer), the code can read any incoming command from the MATLAB application, as well as the encoder and limit switch feedbacks, and then decides the proper state transition for each axis and sends corresponding control signals to the valve control circuit. When an axis is moving and the distance to the target position is within a preset threshold, the axis can be set to braking state, and the motor is driven in the opposite direction to quickly stop the axis. The threshold can be calibrated such that the overshoot is minimized to prevent oscillation around the target position.

In various examples, the controller can be configured to receive signals from sensors (e.g., limit switches, stop buttons in the GUI) to prevent movement of the arm. The triggering of the limit switch or the stop command sent by the user can set the corresponding axis to a braking state if it is in a moving state. For example, once an axis is in the limit-reached state, it can only transition to the moving state in the opposite direction. The signal received by the controller from the sensors (e.g., limit switches) can be used to prevent the robot from traveling beyond the designed motion range and causing collisions or damaging the system. For example, the sensors can be connected via the CPU's hardware interrupt pins and read by interrupt functions in the code so that the state of the corresponding axis is set to braking in the same cycle where the limit switch triggering is read. In another example, the user stop buttons can be used to allow the technician to quickly stop the robot when observing critical situations. In further examples, the signal received by the encoders can be used for velocity monitoring. If the velocity obtained by the encoder is beyond a specified range, the corresponding axis can be set to stop immediately. This is typically triggered by an abnormally low velocity which implies either a large resistive force caused by collisions or potential failures in the robot system.

In some implementations, the robot can be mapped to the geometry of the MRI scanner bore and patient to obtain a safe workspace in robot coordinates. In some aspects, the controller can use signals received from force sensors disposed on the arm member for collision detection.

The controller may include at least one processing unit and system memory to perform arithmetic and logic operations necessary for the operation of the exemplary MR-compatible robot.

The processing unit is configured to execute program code encoded in tangible, computer-readable media. Tangible, computer-readable media refers to any media that is capable of providing data that causes the computing device (i.e., a machine) to operate in a particular fashion, e.g., to control movement of the motion unit of the motorized robotic system or control a setting (e.g., a current) of the instrument. Various computer-readable media may be utilized to provide instructions to the processing unit for execution. Examples of tangible, computer-readable media may include, but is not limited to, volatile media, non-volatile media, removable media, and non-removable media implemented in any method or technology for storage of information such as computer-readable instructions, data structures, program modules or other data.

### Methods of Use

The example systems described herein can overcome issues with conventional MR systems. MRI procedures can require positioning of payloads inside the MRI scanner bore, such as a shim coil for active cardiac shimming. Devices that require manual adjustment, can be time-consuming and cannot achieve scanning with real-time motion control.

The embodiments of the present disclosure described herein can implement a general-purpose MR-compatible robotic cantilever platform for automatic positioning of devices in the bore of an MRI scanner, which can achieve improved efficiency and real-time motion for MRI.

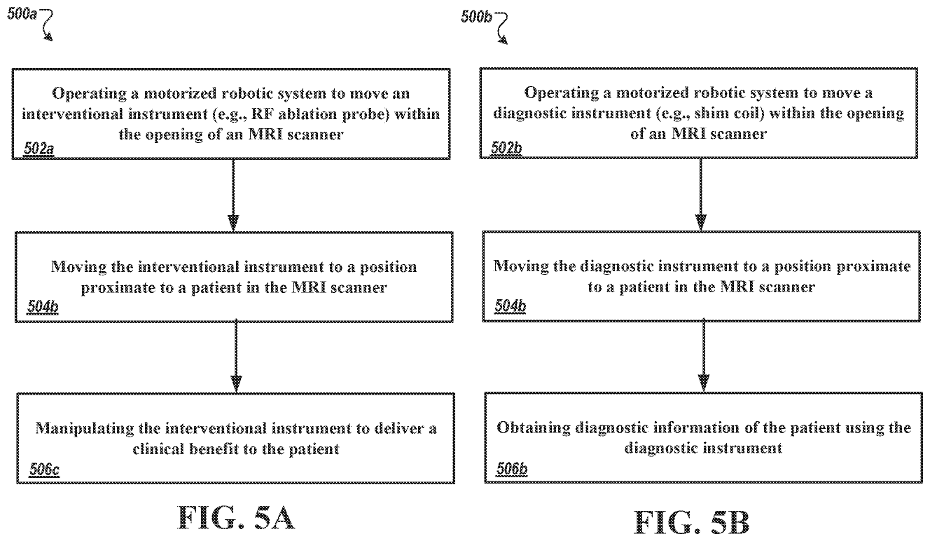

FIGS. 5A-5B each show examples of methods 700a, 700b for manipulating an instrument in an MRI environment using the motorized robotic systems described herein.

In FIG. 5A, the method 500a includes operating a motorized robotic system to move an interventional instrument (e.g., a RF ablation probe) within an opening of an MRI scanner (502a). The method further includes moving the interventional instrument to a position proximate to a patient in the MRI scanner (504a) and manipulating the interventional instrument to deliver a clinical benefit to the patient (506a).

In another example, shown in FIG. 5B, the method 500b includes operating a motorized robotic system to move a diagnostic instrument (e.g., a shim coil) within an opening of an MRI scanner (502b). The method further includes moving the diagnostic instrument to a position proximate to a patient in the MRI scanner (504b) and obtaining diagnostic information of the patient using the diagnostic instrument (506b).

In various aspects, disclosed herein are methods for reducing artifacts in magnetic resonance imaging. In various aspects, the method includes: operating the motorized robot system discussed herein to position the shim coil proximate to a source of distortion (e.g., an implantable cardioverter defibrillator (ICD) or a pacemaker); and supplying a current to the shim coil to produce a controllably distorted magnetic field opposing a magnetic field of the source of distortion. In some aspects, the method includes incrementally moving the shim coil and/or varying the current to the shim coil (e.g., via a feedback loop) to form a distortion field-map (e.g., indicative of an optimal position of the shim coil). Examples of various shim coil controls for reducing artifacts can be found in U.S. Pat. No. 10,338,173, which is hereby incorporated by reference in its entirety.

### Experimental Results and Additional Examples

By way of example, systems and methods for reducing artifacts in magnetic resonance imaging are described.

A motorized robotic system was developed for operation in an MR environment. The study observed various considerations in the development of the MR-compatible system: (i) the system was developed to resist displacement or rotation by the action of the static magnetic field (B₀), the gradient magnetic field or the interaction between those fields and the current in the shim coil (which creates a magnetic field of its own). (ii) Use of the CSS within the bore was restricted to limit additional subject RF (e.g., signal at the MRN scanner Larmor frequency or B₁) heating during MR imaging, relative to the IEC/FDA heating limits (1.5° C./Kg) permitted during human imaging in the scanner. The CSS outer frame also had to be completely insulated from DC or AC electric currents. The system was constructed to meet MR Imaging requirements such that it did not negatively affect the MR Image (iv) Contrast to Noise Ratio or (v) spatial linearity, as measured relative to the scanner performance without the shim system in-place. (vi) Use of commercial MRI surface coils had to be supported.

The CSS performance was specified to (vii) reduce ICD-generated field inhomogeneity on a user-defined 5×5×5 cm³ ROI in the heart from 100 ppm to 25 ppm at all locations, with the ICD-related void locations <10 cm posterior to the patient's abdominal surface. (viii) The imaging improvement should be achieved rapidly, to minimally elongate the duration of the MRI protocol, with the patient typically breath-holding or under General Anesthesia over this time, and (ix) require minimal human interaction within the MRI room during the procedure.

Shim coil for strong directional magnetic field induction. The shim coil design was focused on minimizing the inhomogeneity within a user-specified target ROI (white and black squares in FIG. 6, panels A, B, D). Based upon field maps obtained in humans with ICD's and with ICD's placed on phantoms, it was determined that the inhomogeneity created by ICD was well described by a dipole. The coil winding pattern was obtained by minimizing the inhomogeneity in the target ROI, based upon current segments. It was observed that the optimal current segment configuration could be well approximated by a single shim coil. FIG. 6, panel G shows an example circuit design of the shim coil.

The total magnetic force on the coil was balanced by return currents that were well separated from the central segments (FIG. 6, panel C), effectively creating two coil halves. The B₀ field creates a torque on the two coil halves to fold the coil which is easily compensated by a fiberglass sheet stiffener. To reduce the maximum required current (~10 A) to meet the shimming requirements (75 ppm), 72 coil turns in the central segment were used. The shim coil (FIG. 6, panels E, F) was designed to provide a magnetic field of 0.018 mT/Amp-current at a 15 cm distance from the coil surface. This large current per Amp current should require less than 10 Amperes of direct-(electrical)-current (DC) to correct ICD artifacts deep below the anterior skin to the prescribed extent (75 ppm) over the specified ROI (5×5×5 cm³).

To localize the shim coil location within the bore, a T-shaped fiducial marker was placed 2 cm below the center of the coil. This fiducial marker was filled with Gd-DTPA-doped water, so it could be easily observed with all MRI imaging sequences and used to register the shim coil location in the MRI-scanner coordinate frame. Once this registration was completed, the shim coil could be brought to any desired location using its supporting cantilevered arm.

Penetration-panel electronics prevent RF interference from outside the MRI room. Power to the shim coil was provided by an amplifier in current controlled mode that was located outside the MRI room. To prevent RF noise (RFI) from entering the MRI room, a custom-built multi-stage low-pass RF filter was designed and built to support 15 Ampere currents and placed at the room's penetration panel. The custom filter design was used to ensure stable operation with the current controlled amplifier.

Power amplifier ensures stable current supply to shim coil during MRI imaging. Power to the shim coil was provided by an AE Techron 7224 or 7234 amplifier. The current control feedback loop ensured correction of the output DC current even when it detects induced currents in the shim coil from the MRI scanner's gradient coils, maintaining the net output magnetic field of the shim coil at the prescribed levels.

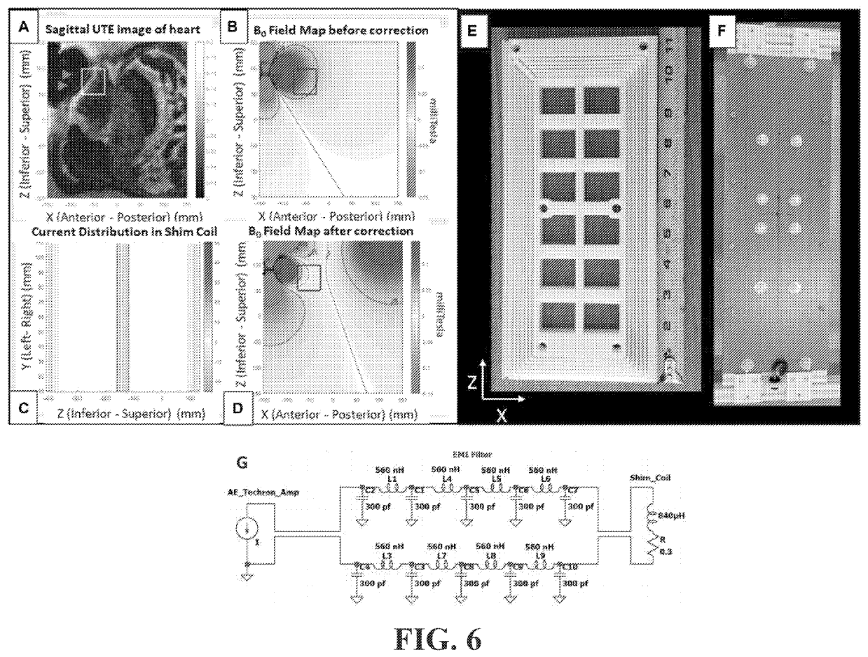

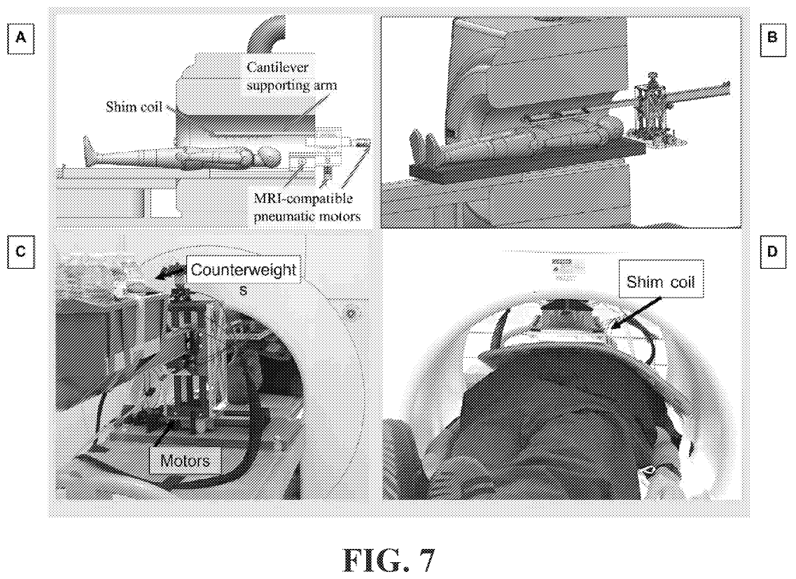

Remotely activated cantilevered shim-coil displacement system to move the shim coil within the bore. The shim coil was attached to a remotely activated 3-stage cantilever platform that enables movements along the scanner X (patient right-left), Y (patient anterior-posterior) and Z (patient superior-inferior) directions in the MRI bore. As shown in FIG. 7, the platform that supports the cantilevered arm was situated immediately outside the scanner bore, with the cantilevered beam protruding up to 180 cm into the bore. The shim coil was suspended from the beam at its distal end. The allowable motion was 140 mm in the X, Y, and Z directions at a 2 mm precision, and the beam supported a 5 kg shim coil. A volume of water was added to the back end of the cantilevered arm, serving as a counterweight, to balance the torque created by the shim coil, which is suspended from the front end of the beam. Linear translation of the beam in 3 directions was provided by lead screws, which were driven by pneumatically actuated motors, each equipped with an optic quadrature encoder for rotation angle feedback and closed-loop control. Chen et al., 2017. The optical fibers for the encoders and the air hoses driving the motors were connected to a control box located outside the MR room via a waveguide, allowing for remote control of the cantilevered arm. The control box was connected to a PC-based (Matlab) program with a user interface designed to enter the desired target position of the shim coil, to the control box, and allow the user to initiate motion. To ensure MR compatibility, the components of the platform were either 3D-printed using PLA and resin, or made of acrylic, nylon, fiberglass, and other plastic materials.

B₀ Field Mapping and optimization of shim coil location and current. Two approaches were used for the optimization of the shim coil location and current. The first approach is deterministic and uses field maps, dipole fitting, and software optimization of shim coil location and current. The second approach is interactive and uses real-time imaging to optimize the shim coil location and current.

For the deterministic approach, static (B₀) Magnetic field maps were acquired using double-echo MRI sequences, with the unwrapped phase difference between the 1^{st} and 2^{nd} echo (TE₁, TE₂) phase-maps, forming the basis for the B₀ field map. They were obtained with conventional cartesian gradient-echo, with a stack of radials self-navigated scan, as well as with ultrashort echo time (UTE) stack of spirals scans. The latter two imaging sequences had a shorter TE, leading to less signal dephasing, as well as a larger frequency bandwidth (>6 KHz), which allowed obtaining correct Bo field maps closer to the largest magnetic field inhomogeneities within the ICD-caused perturbation volume since imaging resulted in volumetrically smaller image voids. The cartesian GRE images were acquired with TE₁=1490 μs and TE₂=1740 μs. The UTE images were acquired with TE₁=50 μs and TE₂=300 μs.

After computing the B₀ Field map, the dipole strength and location were determined. This was done by fitting the dipole field profile to the field map data using Matlab code using a reduced number of points from the field map in the vicinity of the distortion. A coarse grid search was performed before fine tuning the final fitting process.

One advantage of the dipole model is that it allows a determination of the field profile in those regions where there is no MR signal. An example of this is shown in FIG. 6, panel B. To determine the position and current strength of the shim coil, a second step involves locating an ROI on the dipole field map and then optimally fitting the strength and position of the shim coil to reduce the inhomogeneity in the ROI. This process is illustrated in FIG. 6, panel D where the ROI is indicated as a black square, and by comparison with FIG. 6, panel B the field is reduced nearly to zero inside the ROI. The calculations were performed with code written in Matlab.

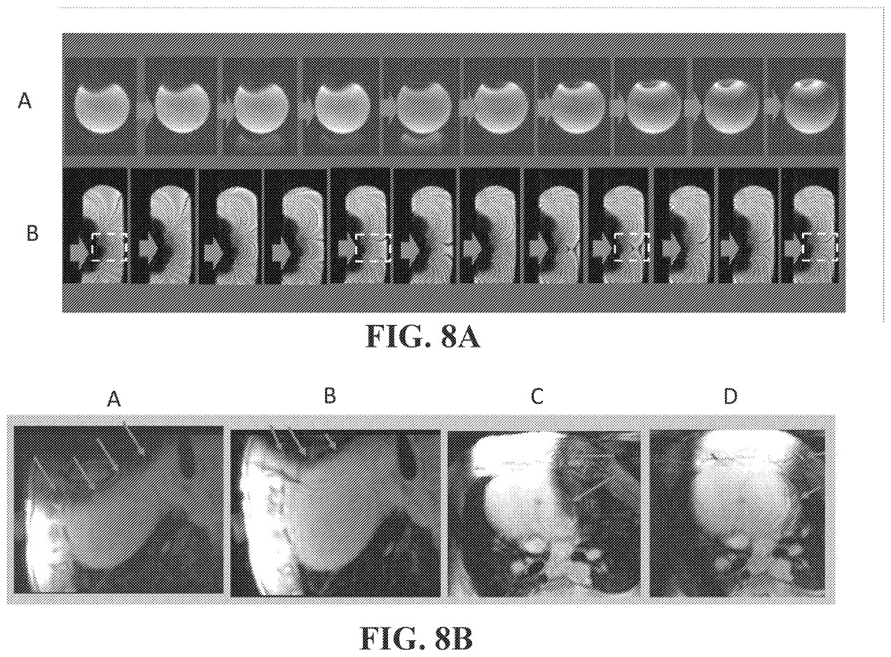

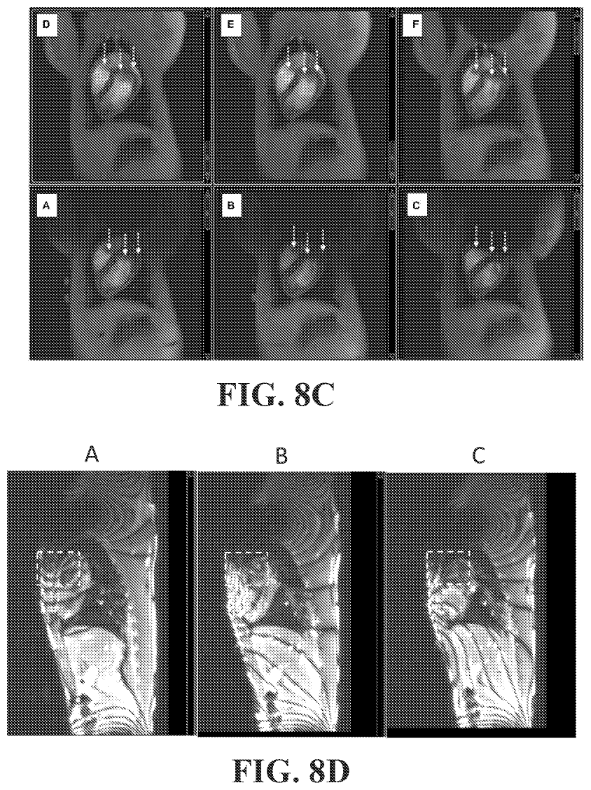

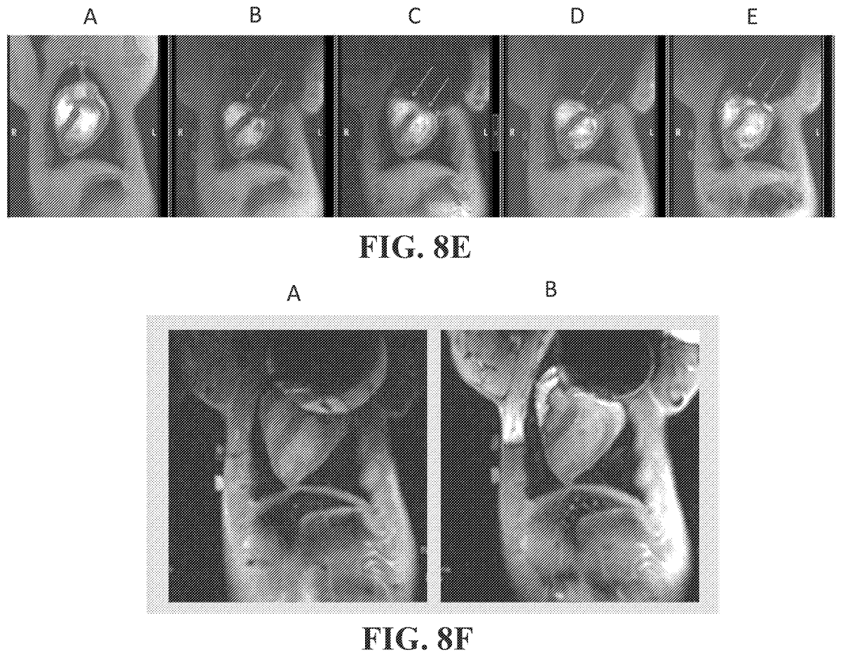

Alternatively, an interactive approach is also possible. Any real-time imaging sequence of a prescribed slice may be set up and observed while the current and position of the shim coil is adjusted during the scanning. This is shown in FIG. 8A. This approach could also be combined with the deterministic approach where the latter approach determines the initial parameters for the shim coil and the interactive approach was used to fine tune the result.

Imaging protocols (sequences, phantoms, animal models) used to validate the system. The study employed 2D and 3D Gradient-Recalled Echo (GRE) sequences, without and with preceding inversion-recovery (IR) pulses, as well as 2D Steady State Free Precession (SSFP) sequences, to validate system performance. ECG-gated single- and multiple-cardiac-phase sequences were used, as well as IR-GRE sequences (LGE) to emulate patient scanning conditions.

Following tests in phantoms, anesthetized swine (N=3, weight 25-35 Kg) were studied, with customary breath-held or respiratory-navigated ECG-gated sequences employed to reduce physiological motion artifacts. The ICD artifacts observed in patients with implanted ICDs were emulated experimentally by placing ICD cases on top of MRI phantoms, and on top of the rib-cages of the swine models.

In the swine (FIG. 8C), the ICD locations were carefully chosen to mirror the location of ICD artifacts in patients, which primarily consist of artifacts at the LV base (e. g. the superior portion of the LV) and on the anterior (i.e. abdominal) portions of the LV wall. Since the chest diameter of young (<0.5 years old) swine is equivalent to that of “skinny” human subjects, the artifacts in swine should mirror those seen in skinny patients, and overestimate those observed in more obese patients.

Medium size Amer. College of Radiology (ACR) resolution phantoms [23] were employed to validate that the insertion of the CSS assembly into the MRI bore, along with execution of MRI sequences with varying parameters, did not reduce the spatial resolution or introduce geometric distortions, relative to scanner performance without the shim coil (i.e. with it outside the bore).

Since the MRI gradients induced currents into the shim coil during the imaging sequence's gradient ramp and falls, MRI imaging was always performed with the CSS power amplifier (AE Techron 7224 or 7234, Elkhart, IN) ON, so that the amplifier's feedback loop could balance out any induced currents. To test for image distortion, two scans with a given sequence were run at the same slice locations, but with the phase-encoding and frequency encoding directions interchanged between successive scans. The resulting images were subtracted to provide a measure of the distortion.

Correcting B₀ inhomogeneity. Optimal artifact reduction involves performing two settings accurately: correctly locating the shim coil and driving the correct current through the shim coil. The combination of both creates the correct magnetic field at the desired inhomogeneity location. Gross corrections were performed by bringing the shim coil to the software prescribed location and driving the recommended current (magnitude and direction) from the power amplifier. Iterative correction of the shim-coil location or current was performed by setting the MRI scanner for continuous (1-2 frames-per-minute) imaging of ECG-gated 2D GRE or 2D SSFP images, while moving the shim coil location in fixed increments, or changing the shim current magnitude in fixed increments.

Balanced SSFP imaging was particularly useful for iterative correction, since SSFP produces very high-contrast images with dark bands occurring at fixed frequency increments (Δf=0.66/TR) when employing sequences with set repetition times (TRs) [24].

Since this Δf also reflects the field inhomogeneity, Δf=[γΔB₀/2π], where γ is the proton gyromagnetic ratio, it is possible to easily visualize the magnetic field inhomogeneity within a given region. Therefore, increasing the spatial distance between adjacent bands is a rapid indication of the improved homogeneity within this area, so we strove to reduce the number of bands within the desired ROI (such as portions of the LV) while iteratively changing the current magnitude or shim coil location.

### Results

MRI Safety. A set of safety and performance tests were performed combining imaging phantoms and the CSS system prior to its approval for use in animal models. These consisted of B1 measurements, SNR measurements, force balance checks, gradient coil coupling, distortion measurements and heating measurements.

The study placed the shim coil alone on the MRI stretcher and validated that the coil did not displace or rotate when inserted the coil into the bore, as well as when current was delivered into the shim coil. Similarly, the study tested the cantilevered system after inserting it into the magnetic field and found that the beam was not displaced by the magnetic field.

Measurements of the reference power and SNR with versus without the CSS system were not significantly different. Gradient coil coupling to the shim coil was evaluated by observing the induced current through the shim coil during an MRI scan. With proper setup of the Techron amplifier the induced current during all MRI sequences studied was negligible.

Although the study did not perform explicit heating tests, signs of additional subject rf heating was not observed during MR imaging. The shim coil assembly was operated for several hours at 10 A continuous current without significant warming to the touch. SAR values and B1 strength were measured to be similar in phantoms with versus without the CSS system present in the bore tube.

To date, phantom and animal testing has been performed with the ICD boxes with lead wires removed. As the leads themselves do not generate any significant magnetic-field artifacts, ICD's used with or without leads should present equivalent magnetic field inhomogeneities. Rf heating of the body due to the lead wires is not a major concern at 1.5 Tesla, which is where we conducted our study, as evidenced by the thousands of ICD patients that have safely undergone MRI imaging at this field strength. We discuss high-field rf heating concerns later on in the Conclusions section.

The CSS outer frame was completely constructed of non-conductive materials, insulating a person who touches it from DC or AC electric voltages or currents.

### Phantom Testing

Measuring image spatial distortion due to CSS. The spatial distortion due to the presence of the CSS system was observed to be primarily along the Y (Anterior-posterior) direction. With the AE Techron 7224 or 7234 amplifier, geometric distortion at 0 Ampere current output from the amplifier was reduced to ~5%, as measured by subtracting GRE images in successive acquisitions with changed phase-encoding directions.

Correcting Bo inhomogeneity interactively in phantoms. Strong artifacts in phantoms were produced by placing ICD cases directly above the phantom. In FIG. 8A, correction of the artifact is performed by increasing the current and observing with real-time imaging the ensuing changes. FIG. 8C, panel B illustrates the role of SSFP's prominent band artifacts in providing a real-time “B₀ field map”, since the distance between adjacent bands in the field maps indicates the level of inhomogeneity.

### Swine Testing

Interactive correction of the B₀ field in swine. After moving the cantilevered shim coil to the B₀ map estimated location, we performed gross changes in the shim current in order to rapidly estimate the current required. FIG. 10 shows how sagittal SSFP images provided a fast approximation of the corrective current required.

2D Cine. Another sequence that permitted rapidly observing improvement in shimming in the swine was execution of 2D ECG-gated multiphase GRE scans during the repositioning of the shim coil location. FIG. 11 shows how improved positioning can provide an LV image similar to the baseline state (with no ICD above the chest), which is shown in FIG. 11, panel A.

2D Coronal LGE. Swine breath-held ECG-gated 2D LGE scans were conducted to validate the ability to correct ICD-related artifacts. Once the shim coil location and current were optimized, it was possible to validate that the shim values were also correct for 2D LGE. FIG. 8E compares 2D LGE images acquired before and after shimming.

3D Navigated LGE imaging was performed in the swine using ECG-gating and prospective respiratory navigators. In FIG. 8B, images can be compared between before and after shimming. Since the scan times for 3D LGE were ~6 minutes, the 3D results demonstrate the temporal stability of the CSS system.

The study created an external shim coil with a current geometry that corrects some but not all, of the dipole field inhomogeneity created by the ferromagnetic ICD box. It is possible to create a local magnetic field that shifts the local Larmor frequency of a user-defined ROI in such a way that the MR spins become visible within that ROI. This is what the current CSS system performs in a workflow-efficient manner since it is able to deliver large currents of >720 AmpereTurns without affecting the performance of the MRI scanner, and as a result of the robotically controlled positioning of the shim coil, it can be brought to any desired location within the bore.

As a limitation, It is important to point out that even small ferromagnetic objects can produce very large magnetic field inhomogeneities in their proximity which the CSS system may not be able to correct. We are currently unable to correct inhomogeneities greater than ~100 ppm, although this may be possible with further development.

The current CSS can correct inhomogeneities ~80 ppm but the site of maximum correction may not overly the desired ROI. We are in the process of optimizing coil positioning by a closed-loop system that 1) optimizes fitting of the CSS field to the image distortion field-map in order to minimize the inhomogeneity measuring the image field distortion before CSS, 2) moves the shim coil to the optimized location using robotic positioning control, and 3) repeating this process until the observed inhomogeneity in the RO1 is minimized.

The study tested the CSS in phantoms and swine. The CSS is unique in that it provides a far larger shimming capability than possible with currently available shim systems. Additionally, the remotely controlled cantilevered displacement of the CSS system is unique in its ability to bring the shim coil to the exact location where it provides maximal inhomogeneity correction. And perform this without requiring the MRI technologists to enter the MRI room.

The CSS system was designed to create a local Bo “step” which only corrects a strong localized inhomogeneity. If it is desired to obtain an image over a larger ROI with less artifacts, then several images can be collected with different currents and position of the shim coil. These images can then be combined or “stitched” to produce the corrected image.

The CSS system can also be used in patients implanted with other invasive active devices, such as spine and brain stimulators.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### Conclusion

The various sizes and dimensions provided herein are merely examples. Other dimensions may be employed.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications, as listed below and throughout this document, describes various application and systems that could be used in combination the exemplary system and are hereby incorporated by reference in their entirety herein.
1. Benjamin, M.M., Sorkness, C.A. Practical and ethical considerations in the management of pacemaker and implantable cardiac defibrillator devices in terminally ill patients Proc Bayl Univ Med Cent 2017; 30: 157-160.
2. Friedman D J, Parzynski C S, Varosy P D, Prutkin J M, Patton K K, Mithani A, Russo A M, Jeptha P. Curtis J P, Al-Khatib S M. Trends and In-Hospital Outcomes Associated with Adoption of the Subcutaneous Implantable Cardioverter Defibrillator in the United States JAMA Cardiol. 2016; 1: 900-911.
3. Kalin R, Stanton M.S. Current clinical issues for MRI scanning of pacemaker and defibrillator patients. Pacing Clin Electrophysiol 2005; 28: 326-328. doi.org/10.1111/j.1540-8159.2005.50024.x
4. Nazarian, S., Reynolds, M.R., Ryan, M.P., Wolff, S.D., Mollenkopf, S.A., Turakhia, M.P. Utilization and likelihood of radiologic diagnostic imaging in patients with implantable cardiac defibrillators. J Magn Reson Imaging 2016; 43: 115-127. doi.org/10.1002/jmri.24971
5. Kalb, B., Indik, J.H., Ott, P., Martin, D.R. MRI of Patients With Implanted Cardiac Devices, JMRI (2018) 47:595-603.doi.org/ 10.1002/jmri.25824
6. Shao, J., Rashid, S., Renella, P., Nguyen, K-L, Hu P. Myocardial T1 Mapping for Patients With Implanted Cardiac Devices Using Wideband Inversion Recovery Spoiled Gradient Echo Readout. Magn. Reson. Med. (2017) 77:1495-1504. doi.org/10.1002/mrm.26223
7. Singh, A., Chen, W., Patel, H.N., Alvi, N., Kawaji, K.D., Besser, S.A., Tung, R., Zou, J., Lang, R.M., Mor-Avi, V., Patel, A.R. Impact of Wideband Late Gadolinium Enhancement Cardiac Magnetic Resonance Imaging on Device-Related Artifacts in Different Implantable Cardioverter-Defibrillator Types, JMRI (2021) 54,1257-1265. doi.org/10.1002/jmri.27608
8. Virani, S.S., Alonso, A., Benjamin, E.J., Bittencourt, M.S., Callaway, C.W., Carson, A.P., Chamberlain, A.M., Chang, A.R., Cheng, S., Delling, F.N., Djousse, L., Elkind, M.S.V., Ferguson, J.F., Fornage, M., Khan, S.S., Kissela, B.M., Knutson, K.L., Kwan, T.W., Lackland, D.T., Lewis, T.T., Lichtman, J.H., Longenecker, C.T., Loop, M.S., Lutsey, P.L., Martin, S.S., Matsushita, K., Moran, A.E., Mussolino, M.E., Perak, A.M., Rosamond, W.D., Roth, G.A., Sampson, U.K.A., Satou, G.M., Schroeder, E.B., Shah, S.H., Shay, C.M., Spartano, N.L., Stokes, A., Tirschwell, D.L., VanWagner, L.B., Tsao, C.W. Heart Disease and Stroke Statistics—2020 Update: A Report From the American Heart Association, Circulation (2020) 141 (9): e139-e596. doi.org/10.1161/CIR.0000000000000757
9. Mahida, S., Sacher, F., Dubois, R., Sermesant, M., Bogun, F., Haïssaguerre, M., Jaïs, P., Cochet, H. Cardiac Imaging in Patients With Ventricular Tachycardia. Circulation. (2017) 136:2491-2507. doi.org/10.1161/CIRCULATIONAHA.117.029349
10. Peck, K.Y., Lim, Y.Z., Hopper, I., Krum, H. Medical therapy versus implantable cardioverter-defibrillator in preventing sudden cardiac death in patients with left ventricular systolic dysfunction and heart failure: a meta-analysis of >35,000 patients. Int J Cardiol. (2014) 1; 173(2): 197-203. doi: 10.1016/j.ijcard.2014.02.014.
11. Mukherjee, R.K., Whitaker, J., Williams, S.E., Razavi, R., O'Neill, M.D. Magnetic resonance imaging guidance for the optimization of ventricular tachycardia ablation. Europace. (2018) 1; 20(11): 1721-1732. doi: 10.1093/europace/euy 040.
12. Chen, Y., Godage, I.S., Tse, Z.T.H., Webster, R.J. 3rd, Barth EJ. Characterization and Control of a Pneumatic Motor for MR-conditional Robotic Applications. IEEE ASME Trans Mechatron. (2017) 22(6):2780-2789. doi: 10.1109/TMECH.2017.2767906.
13. Patel, A.R., Kramer, C.M. Role of Cardiac Magnetic Resonance in the Diagnosis and Prognosis of Nonischemic Cardiomyopathy. JACC Cardiovasc Imaging. (2017) October; 10(10 Pt A): 1180-1193. doi: 10.1016/j.jcmg.2017.08.005.
14. Rashid S, Rapacchi S, Shivkumar K, Plotnik A, Finn J P, Hu P. Modified Wideband Three-Dimensional Late Gadolinium Enhancement MRI for Patients with Implantable Cardiac Devices. Magn. Reson. Med. (2017) 75: 572-584. doi.org/10.1002/mrm.25601
15. Sasaki, T., Hansford, R., Zviman, M.M., Kolandaivelu, A., Bluemke, D.A., Berger, R.D., Calkins, H., Halperin, H.R., Nazarian, S. Quantitative assessment of artifacts on cardiac magnetic resonance imaging of patients with pacemakers and implantable cardioverter-defibrillators. Circ Cardiovasc Imaging. (2011) November; 4(6):662-70. doi: 10.1161/CIRCIMAGING.111.965764.
16. Varma, N., Schaerf, R., Kalbfleisch, S., Pimentel, R., Kroll, M.W., Oza, A. Defibrillation thresholds with right pectoral implantable cardioverter defibrillators and impact of waveform tuning (the Tilt and Tune trial). Europace. 2017 Nov. 1; 19(11):1810-1817. doi: 10.1093/europace/euw306.
17. Choi, S.J., Koch, K.M., Hargreaves, B.A., Stevens, K.J., Gold, G.E. Metal artifact reduction with MAVRIC SL at 3-T MRI in patients with hip arthroplasty. AJR Am J Roentgenol. (2015) January; 204(1): 140-7. doi: 10.2214/AJR.13.11785
18. Zhou, J., Stockmann, J.P., Arango, N., Witzel, T., Scheffler, K., Wald, L.L., Lin, F-H. An orthogonal shim coil for 3T brain imaging. Magn. Reson. Med. (2020) 83(4): 1499-1511. doi.org/10.1002/mrm.28010
19. Winkler, S.A., Schmitt, F., Landes, H., DeBever, J., Wade, T., Alejski, A., Rutt, B.K. Gradient and shim technologies for ultra high field MRI, Neuroimage (2018) 168: 59-70. doi.org/10.1016/j.neuroimage.2016.11.033
20. Han, H., Song, A.W., Truong, T.K. Integrated parallel reception, excitation, and shimming (iPRES). Magn. Reson. Med. (2013) 70(1):241-247. doi.org/10.1002/mrm.24766
21. Yang, H-J., Serry, F., Hu, P., Fan, Z., Shim, H., Christodoulou, A., Wang, N., Kwan, A., Xie, Y., Huang, Y., Stager, J., Liu, W., Azab, L., Handelin, R., Lu, M., Yoosefian, G., Wang, M., Dharmakumar, R., Li, D., Han, H. Ultra-Homogeneous B0 field for High-Field Body Magnetic Resonance Imaging with Unified Shim-RF Coils, (2021) ResearchSquare.com.doi.org/10.21203/rs.3.rs-445400/v1
22. Chen, Y., Godage, I.S., Tse, Z.T.H., Webster, R.J., Barth, E.J. Characterization and control of a pneumatic motor for MR-conditional robotic applications. IEEE/ASME Transactions on Mechatronics 22.6 (2017 ): 2780-2789. doi.org/10.1109/TMECH.2017.2767906
23. ACR, American College of Radiology, Large/Medium Phantom Testing: MRI (Revised Apr. 20 2023). (2023) https://accreditationsupport.acr.org/support/solutions/articles/11000061035-large-medium-phantom-testing-mri-revised-3-27-23. Accessed May 15, 2023.
24. Bieri, O., Scheffler, K. Fundamentals of balanced steady state free precession MRI. J Magn Reson Imaging. (2013) 38:2-11.doi.org/ 10.1002/jmri.24163
25. Saman Nazarian, M.D., Ph.D., Rozann Hansford, R.N., M.P.H., Amir A. Rahsepar, M.D., Valeria Weltin, M.S., Diana McVeigh, B.S., Esra Gucuk Ipek, M.D., Alan Kwan, M.D., Ronald D. Berger, M.D., Ph.D., Hugh Calkins, M.D., Albert C. Lardo, Ph.D., Michael A. Kraut, M.D., Ph.D., Ihab R. Kamel, M.D., Ph.D., Stefan L. Zimmerman, M.D., and Henry R. Halperin, M.D., Safety of Magnetic Resonance Imaging in Patients with Cardiac Devices, N Engl J Med 2017; 377:2555-2564. doi.org/10.1056/NEJMoa1604267.
26. Chen Y, Godage IS, Sengupta S, Liu CL, Weaver KD, Barth EJ. MR-conditional steerable needle robot for intracerebral hemorrhage removal. Int J Comput Assist Radiol Surg. 2019 January; 14(1): 105-115. doi: 10.1007/s11548-018-1854-z. Epub 2018 Sep. 1. PMID: 30173334; PMCID: PMC 7306193.
27. Chen, Yue, Sheng Xu, Alexander Squires, Reza Seifabadi, Ismail Baris Turkbey, Peter A. Pinto, Peter Choyke, Bradford Wood, and Zion Tsz Ho Tse. “MRI-guided robotically assisted focal laser ablation of the prostate using canine cadavers.” IEEE Transactions on Biomedical Engineering 65, no. 7 (2017 ): 1434-1442.
28. Chen, Yue, Joseph Howard, Isuru Godage, and Saikat Sengupta. “Closed loop control of an MR-conditional robot with wireless tracking coil feedback.” Annals of biomedical engineering 47, no. 11(2019 ): 2322-2333.
29. Stoianovici, D., Kim, C., Petrisor, D., Jun, C., Lim, S., Ball, M. W., . . . & Allaf, M. E. (2016). MR safe robot, FDA clearance, safety and feasibility of prostate biopsy clinical trial. IEEE/ASME Transactions on Mechatronics, 22(1), 115-126.
30. Vahid-Araghi, Orang, and Farid Golnaraghi. Friction-induced vibration in lead screw drives. Springer Science & Business Media, 2010

1. A motorized robotic system comprising:
an arm member comprising an elongated beam extending along a longitudinal axis and having a first end and a second end, wherein the first end of the arm member includes an instrument configured to be at least partly positionable within an opening of an MRI scanner having a bore or elongated section, the bore or elongated section having a distal end and a proximal end, and the MRI scanner having the opening of the bore or elongated section at the proximal end; and
a base assembly moveably coupled to the second end of the arm member, the base assembly having a motion unit comprising one or more motors configured to, while a patient is located in the bore or elongated section, (i) move the arm member along the longitudinal axis from (a) a first position having the instrument outside of the bore or elongated section to (b) a second position within the bore or elongated section to place the instrument at or toward the distal end of the bore or elongated section and (ii) move the arm member in a horizontal axis perpendicular to the longitudinal axis and/or a vertical axis at the second position to position the instrument at or around the distal end of the bore or elongated section in relation to the patient.

2. The motorized robotic system of claim 1 further comprising:
a controller, the controller being configured to detect an impact or contact of the instrument or arm member to a wall of the bore or elongated section, to the patient, or an object in the bore or elongated section, and wherein the base assembly, the arm member and/or the instrument includes one or more sensors to provide spatial data to the controller associated with a current position of the arm member or the instrument.

3. The motorized robotic system of claim 2, wherein the controller is further configured to:
receive the spatial data from the one or more sensors; and
control the one or more motors of the motion unit to move the arm member from the current position to a target position.

4. The motorized robotic system of claim 2, wherein the controller is configured to:
receive movement data associated with the patient; and
control the one or more motors to move the arm member based on the patient movement data.

5. The motorized robotic system of claim 1, wherein the arm member and base assembly are substantially free of ferromagnetic and/or paramagnetic materials and comprise an MR-compatible material.

6. The motorized robotic system of claim 1, wherein the base assembly comprises a counterweight system coupled to the arm member at or proximal to the second end, wherein the counterweight system is configured to be dynamically translatable along the longitudinal axis in response to longitudinal movement of the arm member.

7. The motorized robotic system of claim 1, wherein the base assembly comprises a counterweight system coupled to the arm member at or proximal to the second end, wherein the counterweight system comprises a pulley system configured to adaptively stabilized the motorized robotic system as the arm member is translated along the longitudinal axis.

8. The motorized robotic system of claim 1, wherein motion unit comprises one or more lead screws operatively coupled to the one or more motors, wherein actuation of the one or more motors causes the lead screw to rotate such that the arm member translates relative to the base assembly.

9. The motorized robotic system of claim 8, wherein the motion unit comprises a first lead screw positioned along the longitudinal axis, a second lead screw positioned along the horizontal axis, and a third lead screw positioned along the vertical axis.

10. The motorized robotic system of claim 1, wherein the base assembly is disposed on an adjustable carrier, wherein the adjustable carrier is configured to be maneuverable about the horizontal and/or vertical axes to move the base assembly about a height and/or a center of the bore or elongated section.

11. The motorized robotic system of claim 1, wherein the instrument comprises a shim coil configured to generate a magnetic field to create a controlled magnetic field, the shim coil defining an electromagnet secured to the first end of the arm member.

12. The motorized robot system of claim 1, wherein the instrument comprises a second robotic assembly coupled to an ablation instrument, an ultrasound probe, biopsy excising instrument, or fluoroscopy instrument (or other interventional instrument).

13. The motorized robot system of claim 1, wherein the instrument comprises an ablation instrument (RF or thermal), an ultrasound probe, biopsy excising instrument, or fluoroscopy instrument.

14. A method for positioning an instrument within an opening of an MRI scanner, the method comprising:
operating the motorized robot system of claim 1 to move the instrument within the opening of the MRI scanner.

15. A method for reducing artifacts in magnetic resonance imaging, the method comprising:
operating the motorized robot system of claim 11 to position the shim coil proximate to a source of distortion; and
supplying a current to the shim coil to produce a controllably distorted magnetic field opposing a magnetic field of the source of distortion.

16. The method of claim 15, comprising incrementally moving the shim coil and/or varying the current to the shim coil to form a distortion field-map.

17. (canceled)

18. (canceled)

19. (canceled)

20. The system of claim 1, wherein the one or more motors comprises a pneumatic, hydraulic or piezoelectric motor.

21. The system of claim 1, wherein the one or more motors a quadrature encoder.

22. The system of claim 1, further comprising:
a controller, wherein the controller is configured to position the beam relative to the platform with 2 mm positional accuracy.

23. The system of claim 1, further comprising a payload coupled to the beam, wherein the beam is a fiberglass cantilever beam, and wherein the one or more motors are configured to move the beam in two or three degrees of freedom.

24. (canceled)

25. (canceled)

26. (canceled)

27. (canceled)

28. (canceled)

29. (canceled)

30. (canceled)

31. (canceled)

32. (canceled)
